(19) United States
(12) Patent Application Publication
(10) Pub. No.: US 2025/0092447 A1
(43) Pub. Date: Mar. 20, 2025

(54) Methods of Solid-Phase Nucleic Acid Hybridization

(71) Applicant: SomaLogic Operating Co., Inc., Boulder, CO (US)

(72) Inventors: Bhavik Nathwani, Boulder, CO (US); Paul Wilhelm Karl Rothemund, Boulder, CO (US)

(73) Assignee: SomaLogic Operating Co., Inc., Boulder, CO (US)

(21) Appl. No.: 18/888,305

(22) Filed: Sep. 18, 2024

Related U.S. Application Data
(60) Provisional application No. 63/583,836, filed on Sep. 19, 2023.

(51) Int. Cl. C12Q 1/6834; G01N 33/53

(52) U.S. Cl. CPC C12Q 1/6834; G01N 33/5308

(57) ABSTRACT

Methods, devices, reagents and kits for improved hybridization of nucleic acids on a solid phase are provided.

### CROSS-REFERENCE TO RELATED APPLICATIONS

The present application claims the benefit of priority of U.S. Provisional Application No. 63/583,836, filed Sep. 19, 2023, which is incorporated by reference herein in its entirety for any purpose.

### FIELD

The present disclosure relates generally to the field of nucleic acid hybridization, and for example, nucleic acid hybridization on a solid support.

### SUMMARY

Nucleic acid hybridization on a solid support is an integral part of many nucleic acid-based assays, including assays performed on arrays or beads. The present disclosure provides improvements in nucleic acid hybridization on solid supports, including, for example, reduced hybridization times.

Embodiment 1. A method of hybridizing a plurality of solution-phase nucleic acids to a plurality of solid-phase nucleic acids, comprising contacting the solid-phase nucleic acids with a hybridization solution comprising the solution-phase nucleic acids and a hybridization catalyst, wherein the hybridization catalyst comprises a polycationic main chain with hydrophilic side chains.

Embodiment 2. The method of embodiment 1, wherein the plurality of solution-phase nucleic acids comprises at least 100, at least 500, at least 1000, at least 2000, at least 3000, or at least 5000 different nucleic acids having different nucleic acid sequences.

Embodiment 3. The method of embodiment 1 or embodiment 2, wherein the plurality of solid-phase nucleic acids comprises at least 100, at least 500, at least 1000, at least 2000, at least 3000, or at least 5000 different nucleic acids having different nucleic acid sequences.

Embodiment 4. The method of any one of embodiments 1-3, wherein the solid-phase nucleic acids are bound to an array, flow cell, or beads.

Embodiment 5. The method of any one of embodiments 1-3, wherein the solid-phase nucleic acids are bound to an array.

Embodiment 6. The method of any one of embodiments 1-5, wherein the hybridization solution comprises 100-300 mM monovalent cationic salt.

Embodiment 7. The method of embodiment 6, wherein the monovalent cationic salt is NaCl or sodium citrate.

Embodiment 8. The method of any one of embodiments 1-7, wherein the hybridization catalyst comprises a polylysine, polyarginine, or polyornithine main chain.

Embodiment 9. The method of any one of embodiments 1-8, wherein the hybridization catalyst comprises polyethylene glycol, polyacrylamide, polyvinyl alcohol, or dextran side chains.

Embodiment 10. The method of any one of embodiments 1-9, wherein the hybridization catalyst comprises a polylysine main chain and polyethylene glycol (PEG) side chains.

Embodiment 11. The method of embodiment 10, wherein the hybridization catalyst comprises 10-500 or 10-300 or 20-200 lysines in the polylysine main chain; 5-50% or 5-40% or 5-30% or 10-30% PEG modification, and/or wherein the PEG has an average molecular weight of 1-20 kDa or 2-10 kDa, or 2-8 kDa, or about 5 kDa.

Embodiment 12. The method of any one of embodiments 1-11, wherein the hybridization catalyst is present in an amount such that the ratio of number of cations in the polycationic backbone of the hybridization catalyst to the number of phosphates in the plurality of solution-phase nucleic acids and plurality of solid-phase nucleic acids (N:P ratio) is between 1:1 and 200:1, or between 1:1 and 100:1, or between 1:1 and 50:1, or between 1:1 and 25:1.

Embodiment 13. The method of any one of the preceding embodiments, wherein hybridization of the solution-phase nucleic acids to the solid-phase nucleic acids is at least 2-fold, at least 3-fold, at least 4-fold, or at least 5-fold faster in the presence of the hybridization catalyst than in the absence of the hybridization catalyst.

Embodiment 14. The method of any one of embodiments 1-13, wherein the solution-phase nucleic acids comprise RNA, DNA, genomic DNA, mitochondrial DNA, mRNA, microRNA, aptamers, and/or modified oligonucleotides.

Embodiment 15. The method of any one of embodiments 1-14, wherein the method of hybridization is a step in an assay selected from next-generation sequencing, diagnostic microarrays (such as those for detecting specific genetic mutations, chromosomal aberrations, epigenetic changes, SNPs, etc.), gene expression microarrays, microsatellite analysis, proteomic assays, and strand-displacement assays.

Embodiment 16. The method of any one of embodiments 1-15, wherein the solution-phase nucleic acids are conjugated to non-nucleic acid moiety.

Embodiment 17. The method of embodiment 16, wherein the non-nucleic acid moiety is selected from a protein, an antibody, a lipid, and a small molecule.

Embodiment 18. The method of any one of embodiments 1-17, wherein the solution-phase nucleic acids each comprise a barcode sequence.

Embodiment 19. The method of any one of embodiments 1-18, wherein the solution-phase nucleic acids are not amplified prior to hybridization to the solid-phase nucleic acids.

Embodiment 20. The method of any one of embodiments 1-18, wherein the solution-phase nucleic acids are amplified prior to hybridization to the solid-phase nucleic acids.

Embodiment 21. The method of embodiment 20, wherein the amplification comprises PCR, RT-PCR, or qPCR.

Embodiment 22. The method of any one of embodiment 1-21, wherein the solution-phase nucleic acids are aptamers.

Embodiment 23. The method of embodiment 22, wherein the aptamers are slow off-rate aptamers.

Embodiment 24. The method of embodiment 22 or embodiment 23, wherein each aptamer comprises at least one, at least two, at least three, or at least five C-5 modified pyrimidines.

Embodiment 25. The method of embodiment 24, wherein each C-5 modified pyrimidine comprises a C-5 modified uracil base or a C-5 modified cytosine base.

Embodiment 26. The method of any one of embodiments 22-25, wherein each aptamer is capable of binding to a protein target.

Embodiment 27. The method of any one of embodiments 21-25, wherein prior to hybridization to the solid-phase nucleic acids, the aptamers were eluted from aptamer/protein complexes.

Embodiment 28. The method of embodiment 27, wherein the aptamer/protein complexes were formed by contacting the aptamers with a biological sample comprising proteins.

Embodiment 29. A method of detecting a plurality of protein targets in a biological sample, comprising contacting the biological sample with a plurality of aptamers, wherein each aptamer is a nucleic acid, and wherein each aptamer specifically binds a protein target, to form a plurality of aptamer/protein complexes, releasing the bound aptamers from the aptamer/protein complexes into solution to form a plurality of solution-phase nucleic acids, and hybridizing the plurality of solution-phase nucleic acids to a plurality of solid-phase nucleic acids according to the method of any one of embodiments 1-28, and detecting the hybridized solution-phase nucleic acids.

Embodiment 30. The method of embodiment 28 or embodiment 29, wherein the biological sample is selected from plasma, serum, urine, whole blood, leukocytes, peripheral blood mononuclear cells, buffy coat, sputum, tears, mucus, nasal washes, nasal aspirate, semen, saliva, peritoneal washings, ascites, cystic fluid, meningeal fluid, amniotic fluid, glandular fluid, lymph fluid, nipple aspirate, bronchial aspirate, bronchial brushing, synovial fluid, joint aspirate, organ secretions, cells, a cellular extract, and cerebrospinal fluid.

### BRIEF DESCRIPTION OF THE DRAWINGS

FIG. 1 illustrates certain exemplary 5-position modified uridines and cytidines that may be incorporated into aptamers.

FIG. 2 illustrates certain exemplary modifications that may be present at the 5-position of uridine. The chemical structure of the C-5 modification includes the exemplary amide linkage that links the modification to the 5-position of uridine. The 5-position moieties shown include two phenyl groups covalently attached to one another. The 5-position moieties shown include a phenylbenzyl moiety (e.g., BPE, PBnd, DBM), a 4-phenoxybenzyl moiety (e.g., POP), a diphenylpropyl moiety (e.g., DPP), a benzhydryl moiety (e.g., BH).

FIG. 3 illustrates certain exemplary modifications that may be present at the 5-position of cytidine. The chemical structure of the C-5 modification includes the exemplary amide linkage that links the modification to the 5-position of cytidine. The 5-position moieties shown include two phenyl groups covalently attached to one another. The 5-position moieties shown include a phenylbenzyl moiety (e.g., BPE, PBnd, DBM), a 4-phenoxybenzyl moiety (e.g., POP), a diphenylpropyl moiety (e.g., DPP), a benzhydryl moiety (e.g., BH).

FIG. 4 illustrates certain exemplary modifications that may be present at the 5-position of uridine. The chemical structure of the C-5 modification includes the exemplary amide linkage that links the modification to the 5-position of the uridine. The 5-position moieties shown include a benzyl moiety (e.g., Bn, PE and a PP), a naphthyl moiety (e.g., Nap, 2Nap, NE), a butyl moiety (e.g, iBu), a fluorobenzyl moiety (e.g., FBn), a tyrosyl moiety (e.g., a Tyr), a 3,4-methylenedioxy benzyl (e.g., MBn), a morpholino moiety (e.g., MOE), a benzofuranyl moiety (e.g., BF), an indole moiety (e.g, Trp) and a hydroxypropyl moiety (e.g., Thr).

FIG. 5 illustrates certain exemplary modifications that may be present at the 5-position of cytidine. The chemical structure of the C-5 modification includes the exemplary amide linkage that links the modification to the 5-position of the cytidine. The 5-position moieties shown include a benzyl moiety (e.g., Bn, PE and a PP), a naphthyl moiety (e.g., Nap, 2Nap, NE, and 2NE) and a tyrosyl moiety (e.g., a Tyr).

FIG. 6 provides an example overview of the dilution sets for a biological sample, the corresponding capture reagent sets for their respective dilutions, and the general overview of the sequential two-catch system (catch-1 and catch-2). Three different dilution groups may be created from a biological sample that includes a Z % dilution of the biological sample or DIL3, a Y % dilution of the biological sample or DIL2 and a X % dilution of the biological sample or DIL1, where Z is greater than Y, and Y is greater than X (or Z is a greater dilution than the Y dilution, and the Y dilution is a greater dilution than the X dilution). Each dilution has its own set of corresponding capture reagents (A3 for DIL1, A2 for DIL2 and A1 for DIL3) that bind to a specific set of proteins.

FIG. 7 provides an overview of the three different dilution groups of plasma that were made: a 0.005% dilution (DIL1), a 0.5% dilution (DIL2) and a 20% dilution (DIL3), where the relative high, medium and low abundance proteins were measured, respectively. Further, the aptamer sets for each of DIL1, DIL2 and DIL3 were A1, A2 and A3, respectively. The A3 group of aptamers had 4,271 different aptamers (or ˜81% of the total number of aptamers), the A2 group had 828 different aptamers (or ˜ 16% of the total number of aptamers) and the A1 group has 173 different aptamers (˜3% of the total number of aptamers) for a total of 5,272 different aptamers. The three different dilution sets were transferred sequentially from the catch-1 step of the assay to the catch-2 step of the assay.

FIG. 8 provides an example overview of the dilution sets for a biological sample, the corresponding capture reagent sets for their respective dilutions, and the general overview of the two-catch system (catch-1 and catch-2). Two different dilution groups may be created from a biological sample that includes a Z % dilution of the biological sample or DIL4 and an X % dilution of the biological sample or DIL1, where Z is greater than X (or Z is a greater dilution than the X dilution). Each dilution has its own set of corresponding capture reagents (A3 for DIL1 and A1 for DIL4) that bind to a specific set of proteins. The two different dilution sets were transferred sequentially from the catch-1 step of the assay to the catch-2 step of the assay.

FIG. 9 shows a plot of signal intensity from hybridization of SOMAmers on SomaArray plates in the presence of hybridization catalyst provided herein at various N:P ratios (ratio of the number of amines on the catalyst to the number of phosphates on the nucleic acid backbone) (“magic buffer”) versus hybridization in the absence of hybridization catalyst (x-axis).

FIG. 10 shows a plot of the frequency of coefficients of variance of about 7,000 SOMAmers hybridized on a SomaArray plate in the presence or absence of a hybridization catalyst provided herein.

FIG. 11 shows a plot of the frequency of coefficients of variance of hybridization to sequences on a SomaArray plate that were not complementary to any SOMAmers in the assay, in the presence or absence of a hybridization catalyst provided herein.

FIG. 12 shows repeatability of hybridization of SOMAmers on SomaArray plates in the presence of hybridization catalyst.

FIG. 13 shows hybridization of high-concentration SOMAmers on SomaArray plates in the presence of hybridization catalyst aged for 2 weeks or 4 weeks.

FIG. 14 shows hybridization of low-concentration SOMAmers on SomaArray plates in the presence of hybridization catalyst aged for 2 weeks or 4 weeks.

FIG. 15 shows hybridization of SOMAmers to a custom CGH slide microarray (Agilent, part number G4885A) in the presence and absence of hybridization catalyst provided herein, with an incubation time of 19 hours.

FIG. 16 shows a plot of the cumulative distribution function of SOMAmers against the measured coefficient of variance (CV). The left shift of the blue curve (with magic buffer) indicates a lower CV compared to the SOP buffer.

### DETAILED DESCRIPTION

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Further, ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of” mean±20% of the indicated range, value, or structure, unless otherwise indicated. As used herein, the terms “include” and “comprise” are open ended and are used synonymously.

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

As used herein, the term “nucleotide” refers to a ribonucleotide or a deoxyribonucleotide, or a modified form thereof, as well as an analog thereof. Nucleotides include species that include purines (e.g., adenine, hypoxanthine, guanine, and their derivatives and analogs) as well as pyrimidines (e.g., cytosine, uracil, thymine, and their derivatives and analogs). As used herein, the term “cytidine” is used generically to refer to a ribonucleotide, deoxyribonucleotide, or modified ribonucleotide comprising a cytosine base, unless specifically indicated otherwise. The term “cytidine” includes 2′-modified cytidines, such as 2′-fluoro, 2′-methoxy, etc. Similarly, the term “modified cytidine” or a specific modified cytidine also refers to a ribonucleotide, deoxyribonucleotide, or modified ribonucleotide (such as 2′-fluoro, 2′-methoxy, etc.) comprising the modified cytosine base, unless specifically indicated otherwise. The term “uridine” is used generically to refer to a ribonucleotide, deoxyribonucleotide, or modified ribonucleotide comprising a uracil base, unless specifically indicated otherwise. The term “uridine” includes 2′-modified uridines, such as 2′-fluoro, 2′-methoxy, etc. Similarly, the term “modified uridine” or a specific modified uridine also refers to a ribonucleotide, deoxyribonucleotide, or modified ribonucleotide (such as 2′-fluoro, 2′-methoxy, etc.) comprising the modified uracil base, unless specifically indicated otherwise.

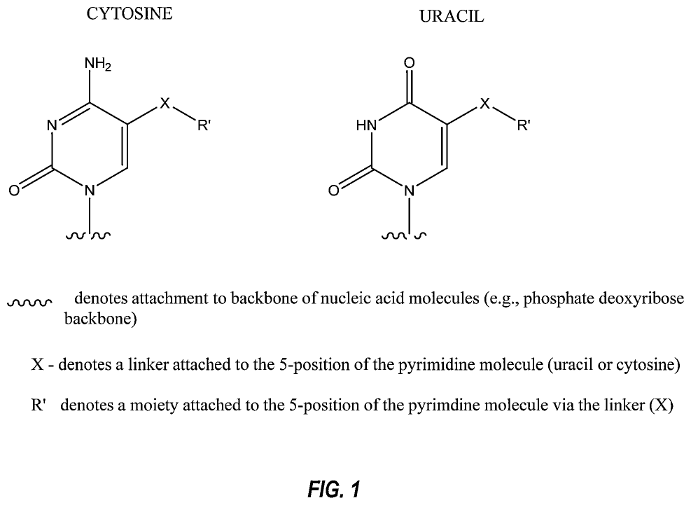

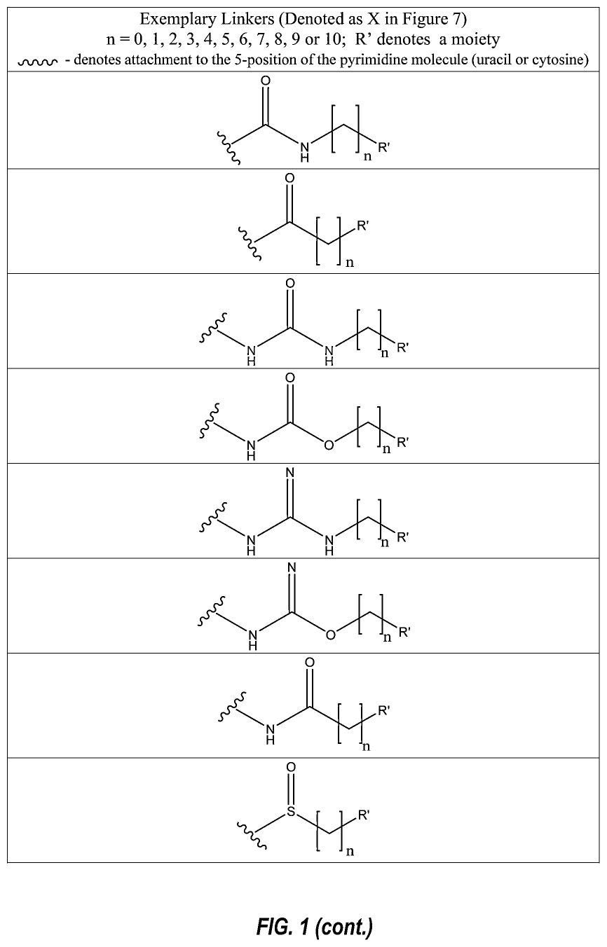

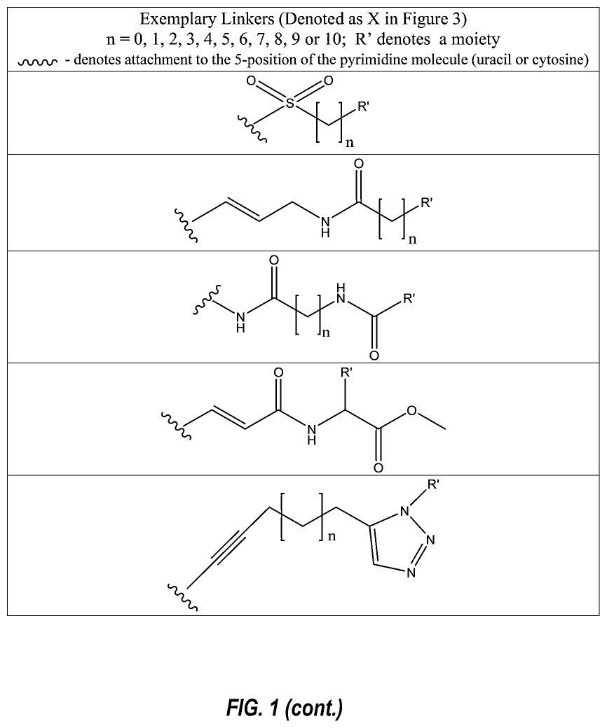

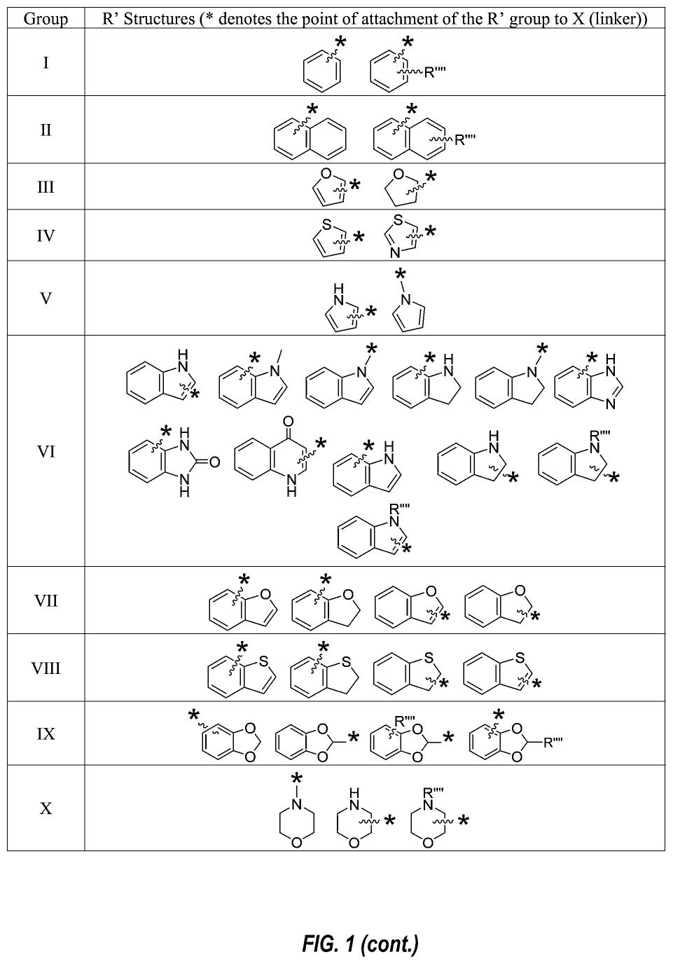

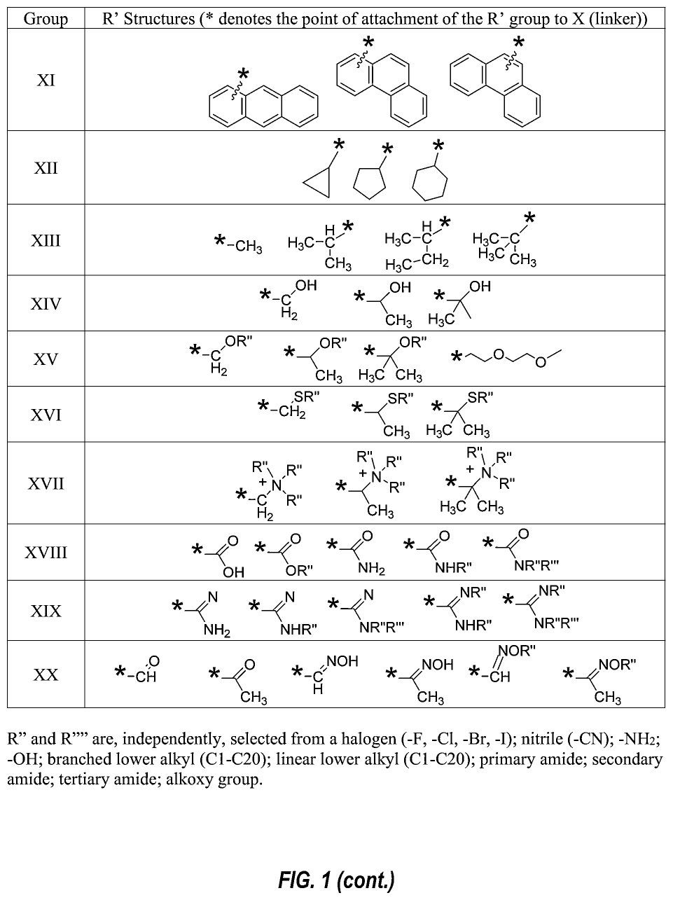

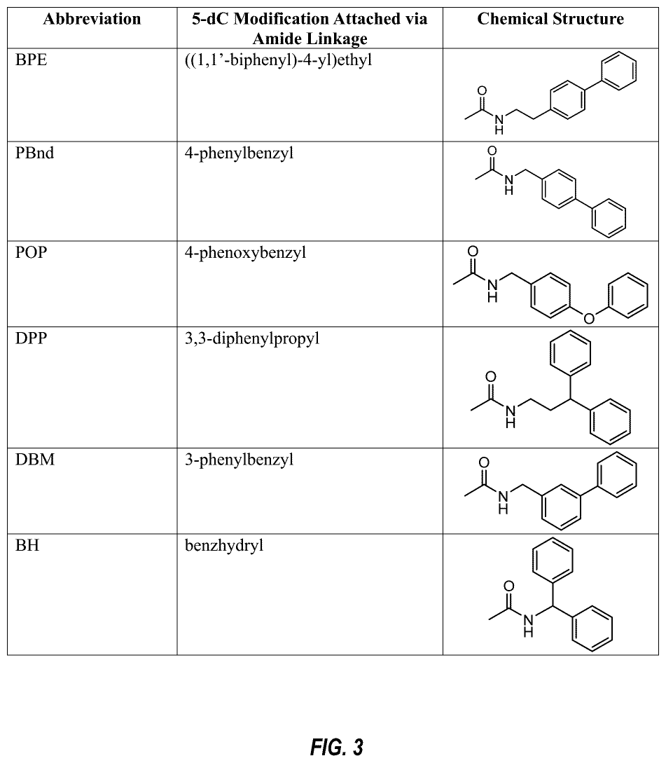

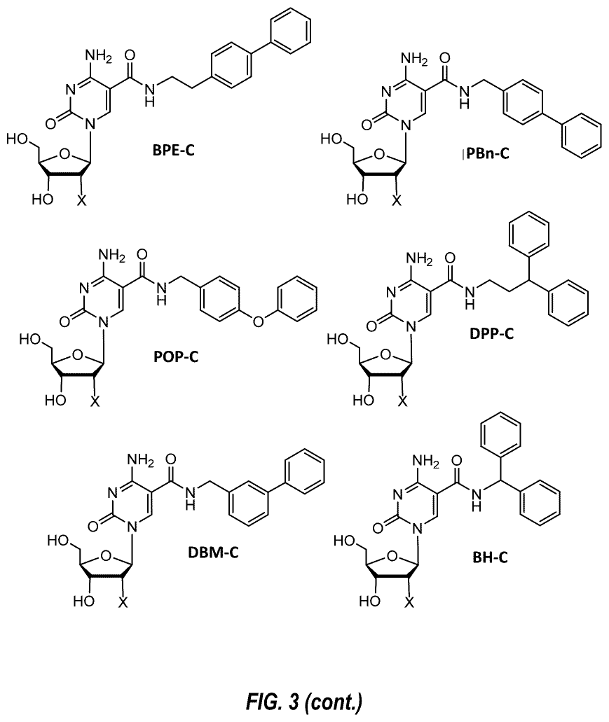

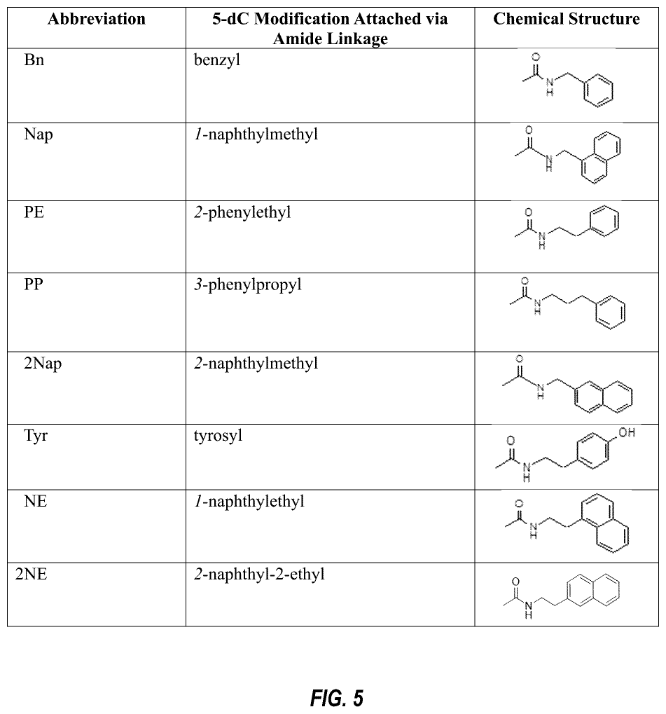

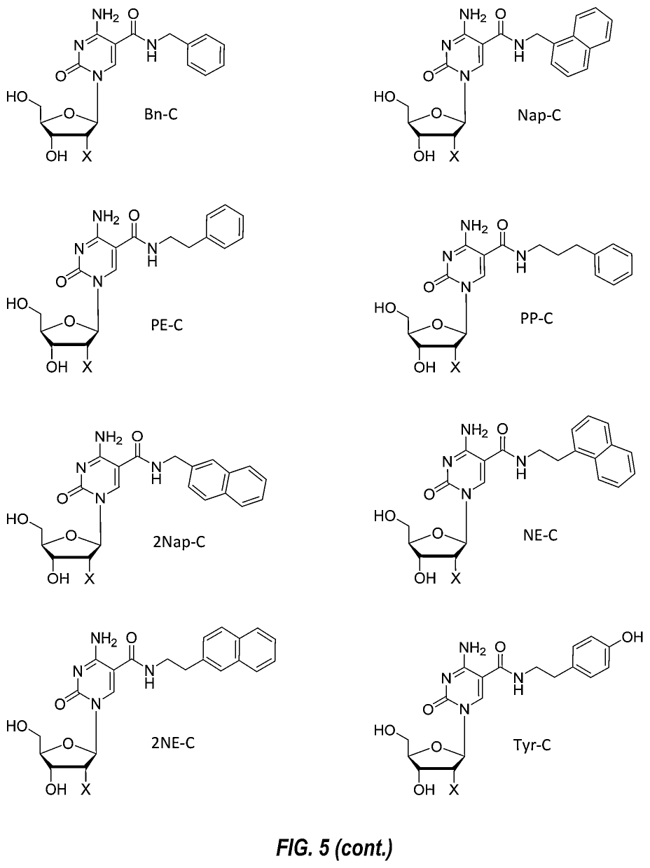

As used herein, the term “C-5 modified carboxamidecytidine” or “cytidine-5-carboxamide” or “5-position modified cytidine” or “C-5 modified cytidine” refers to a cytidine with a carboxyamide (—C(O)NH—) modification at the C-5 position of the cytidine including, but not limited to, those moieties (R′) illustrated herein. See, e.g., FIGS. 1, 3, and 5. Exemplary C-5 modified carboxamidecytidines include, but are not limited to, 5-(N-((1,1′-biphenyl)-4-yl)ethyl)-2′-deoxycytidine (referred to as “BPEdC” and shown in FIG. 3), 5-(N-4-phenylbenzyl)-2′-deoxycytidine (referred to as “PBnddC” and shown in FIG. 3), 5-(N-4-phenoxybenzyl)-2′-deoxycytidine (referred to as “POPdC” and shown in FIG. 3), 5-(N-3,3-diphenylpropyl)-2′-deoxycytidine (referred to as “DPPdC” and shown in FIG. 3), 5-(N-3-phenylbenzyl)-2′-deoxycytidine (referred to as “DBMdC” and shown in FIG. 3), 5-(N-benzhydryl)-2′-deoxycytidine (referred to as “BHdC” and shown in FIG. 3), 5-(N-benzylcarboxamide)-2′-deoxycytidine (referred to as “BndC” and shown in FIG. 5); 5-(N-2-phenylethylcarboxamide)-2′-deoxycytidine (referred to as “PEdC” and shown in FIG. 5); 5-(N-3-phenylpropylcarboxamide)-2′-deoxycytidine (referred to as “PPdC” and shown in FIG. 5); 5-(N-1-naphthylmethylcarboxamide)-2′-deoxycytidine (referred to as “NapdC” and shown in FIG. 5); 5-(N-2-naphthylmethylcarboxamide)-2′-deoxycytidine (referred to as “2NapdC” and shown in FIG. 5); 5-(N-1-naphthyl-2-ethylcarboxamide)-2′-deoxycytidine (referred to as “NEdC” and shown in FIG. 5); 5-(N-2-naphthyl-2-ethylcarboxamide)-2′-deoxycytidine (referred to as “2NEdC” and shown in FIG. 5); and 5-(N-tyrosylcarboxyamide)-2′-deoxycytidine (referred to as TyrdC and shown in FIG. 5). In some embodiments, the C5-modified cytidines, e.g., in their triphosphate form, are capable of being incorporated into an oligonucleotide by a polymerase (e.g., KOD DNA polymerase).

Chemical modifications of the C-5 modified cytidines described herein can also be combined with, singly or in any combination, 2′-position sugar modifications, modifications at exocyclic amines, and substitution of 4-thiocytidine and the like.

As used herein, the term “C-5 modified uridine” or “5-position modified uridine” refers to a uridine (typically a deoxyuridine) with a carboxyamide (—C(O)NH—) modification at the C-5 position of the uridine, e.g., as shown in FIG. 1. In some embodiments, the C5-modified uridines, e.g., in their triphosphate form, are capable of being incorporated into an oligonucleotide by a polymerase (e.g., KOD DNA polymerase). Nonlimiting exemplary 5-position modified uridines include:
5-(N-((1,1′-biphenyl)-4-yl)ethyl)-2′-deoxyuridine (BPEdU)
5-(N-4-phenylbenzyl)-2′-deoxyuridine (PBnddU)
5-(N-4-phenoxybenzyl)-2′-deoxyuridine (POPdU)
5-(N-3,3-diphenylpropyl)-2′-deoxyuridine (DPPdU)
5-(N-3-phenylbenzyl)-2′-deoxyuridine (DBMdU)
5-(N-benzhydryl)-2′-deoxyuridine (BHdU)
5-(N-benzylcarboxyamide)-2′-deoxyuridine (BndU),
5-(N-benzylcarboxyamide)-2′-O-methyluridine,
5-(N-benzylcarboxyamide)-2′-fluorouridine,
5-(N-phenethylcarboxyamide)-2′-deoxyuridine (PEdU),
5-(N-thiophenylmethylcarboxyamide)-2′-deoxyuridine (ThdU),
5-(N-isobutylcarboxyamide)-2′-deoxyuridine (iBudU),
5-(N-tyrosylcarboxyamide)-2′-deoxyuridine (TyrdU),
5-(N-3,4-methylenedioxybenzylcarboxyamide)-2′-deoxyuridine (MBndU),
5-(N-4-fluorobenzylcarboxyamide)-2′-deoxyuridine (FBndU),
5-(N-3-phenylpropylcarboxyamide)-2′-deoxyuridine (PPdU),
5-(N-imidizolylethylcarboxyamide)-2′-deoxyuridine (ImdU),
5-(N-isobutylcarboxyamide)-2′-O-methyluridine,
5-(N-isobutylcarboxyamide)-2′-fluorouridine,
5-(N-tryptaminocarboxyamide)-2′-deoxyuridine (TrpdU),
5-(N-R-threoninylcarboxyamide)-2′-deoxyuridine (ThrdU),
5-(N-tryptaminocarboxyamide)-2′-O-methyluridine,
5-(N-tryptaminocarboxyamide)-2′-fluorouridine,
5-(N-[1-(3-trimethylamonium) propyl]carboxyamide)-2′-deoxyuridine chloride,
5-(N-naphthylmethylcarboxyamide)-2′-deoxyuridine (NapdU),
5-(N-naphthylmethylcarboxyamide)-2′-O-methyluridine,
5-(N-naphthylmethylcarboxyamide)-2′-fluorouridine,
5-(N-[1-(2,3-dihydroxypropyl)]carboxyamide)-2′-deoxyuridine),
5-(N-2-naphthylmethylcarboxyamide)-2′-deoxyuridine (2NapdU),
5-(N-2-naphthylmethylcarboxyamide)-2′-O-methyluridine,
5-(N-2-naphthylmethylcarboxyamide)-2′-fluorouridine,
5-(N-1-naphthylethylcarboxyamide)-2′-deoxyuridine (NEdU),
5-(N-1-naphthylethylcarboxyamide)-2′-O-methyluridine,
5-(N-1-naphthylethylcarboxyamide)-2′-fluorouridine,
5-(N-2-naphthylethylcarboxyamide)-2′-deoxyuridine (2NEdU),
5-(N-2-naphthylethylcarboxyamide)-2′-O-methyluridine,
5-(N-2-naphthylethylcarboxyamide)-2′-fluorouridine,
5-(N-3-benzofuranylethylcarboxyamide)-2′-deoxyuridine (BFdU),
5-(N-3-benzofuranylethylcarboxyamide)-2′-O-methyluridine,
5-(N-3-benzofuranylethylcarboxyamide)-2′-fluorouridine,
5-(N-3-benzothiophenylethylcarboxyamide)-2′-deoxyuridine (BTdU),
5-(N-3-benzothiophenylethylcarboxyamide)-2′-O-methyluridine, and
5-(N-3-benzothiophenylethylcarboxyamide)-2′-fluorouridine.

As used herein, the terms “modify,” “modified,” “modification,” and any variations thereof, when used in reference to an oligonucleotide, means that at least one of the four constituent nucleotide bases (i.e., A, G, T/U, and C) of the oligonucleotide is an analog or ester of a naturally occurring nucleotide. In some embodiments, the modified nucleotide confers nuclease resistance to the oligonucleotide. Additional modifications can include backbone modifications, methylations, unusual base-pairing combinations such as the isobases isocytidine and isoguanidine, and the like. Modifications can also include 3′ and 5′ modifications, such as capping. Other modifications can include substitution of one or more of the naturally occurring nucleotides with an analog, internucleotide modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present on the sugar of a nucleotide may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, polyethylene glycol (PEG) polymers in one embodiment ranging from about 10 to about 80 kDa, PEG polymers in another embodiment ranging from about 20 to about 60 kDa, or other hydrophilic or hydrophobic biological or synthetic polymers.

As used herein, “nucleic acid,” “oligonucleotide,” and “polynucleotide” are used interchangeably to refer to a polymer of nucleotides and include DNA, RNA, DNA/RNA hybrids and modifications of these kinds of nucleic acids, oligonucleotides and polynucleotides, wherein the attachment of various entities or moieties to the nucleotide units at any position are included. The terms “polynucleotide,” “oligonucleotide,” and “nucleic acid” include double- or single-stranded molecules as well as triple-helical molecules. Nucleic acid, oligonucleotide, and polynucleotide are broader terms than the term aptamer and, thus, the terms nucleic acid, oligonucleotide, and polynucleotide include polymers of nucleotides that are aptamers but the terms nucleic acid, oligonucleotide, and polynucleotide are not limited to aptamers.

Polynucleotides can also contain analogous forms of ribose or deoxyribose sugars that are generally known in the art, including 2′-O-methyl, 2′-O-allyl, 2′-O-ethyl, 2′-O-propyl, 2′-O—CH₂CH₂OCH₃, 2′-fluoro, 2′-NH₂ or 2′-azido, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. As noted herein, one or more phosphodiester linkages may be replaced by alternative linking groups. These alternative linking groups include embodiments wherein phosphate is replaced by P(O)S (“thioate”), P(S)S (“dithioate”), (O)NR^{X}₂ (“amidate”), P(O)R^{X}, P(O)OR^{X}, CO or CH₂ (“formacetal”), in which each R^{X} or R^{X}′ are independently H or substituted or unsubstituted alkyl (C1-C20) optionally containing an ether (—O—) linkage, aryl, alkenyl, cycloalkyl, cycloalkenyl or araldyl. Not all linkages in a polynucleotide need be identical. Substitution of analogous forms of sugars, purines, and pyrimidines can be advantageous in designing a final product, as can alternative backbone structures like a polyamide backbone, for example.

Polynucleotides can also contain analogous forms of carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside.

If present, a modification to the nucleotide structure can be imparted before or after assembly of a polymer. A sequence of nucleotides can be interrupted by non-nucleotide components. A polynucleotide can be further modified after polymerization, such as by conjugation with a labeling component.

As used herein, the term “at least one nucleotide” when referring to modifications of a nucleic acid, refers to one, several, or all nucleotides in the nucleic acid, indicating that any or all occurrences of any or all of A, C, T, G or U in a nucleic acid may be modified or not.

As used herein, “nucleic acid ligand,” “aptamer,” “SOMAmer,” “modified aptamer,” and “clone” are used interchangeably to refer to a non-naturally occurring nucleic acid that has a desirable action on a target molecule. A desirable action includes, but is not limited to, binding of the target, catalytically changing the target, reacting with the target in a way that modifies or alters the target or the functional activity of the target, covalently attaching to the target (as in a suicide inhibitor), and facilitating the reaction between the target and another molecule. In one embodiment, the action is specific binding affinity for a target molecule, such target molecule being a three dimensional chemical structure other than a polynucleotide that binds to the aptamer through a mechanism which is independent of Watson/Crick base pairing or triple helix formation, wherein the aptamer is not a nucleic acid having the known physiological function of being bound by the target molecule. Aptamers to a given target include nucleic acids that are identified from a candidate mixture of nucleic acids, where the aptamer is a ligand of the target, by a method comprising: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers of the target molecule are identified. It is recognized that affinity interactions are a matter of degree; however, in this context, the “specific binding affinity” of an aptamer for its target means that the aptamer binds to its target generally with a much higher degree of affinity than it binds to other, non-target, components in a mixture or sample. An “aptamer,” “SOMAmer,” or “nucleic acid ligand” is a set of copies of one type or species of nucleic acid molecule that has a particular nucleotide sequence. An aptamer can include any suitable number of nucleotides. “Aptamers” refer to more than one such set of molecules. Different aptamers can have either the same or different numbers of nucleotides. Aptamers may be DNA or RNA and may be single stranded, double stranded, or contain double stranded or triple stranded regions. In some embodiments, the aptamers are prepared using a SELEX process as described herein, or known in the art.

As used herein, a “SOMAmer” or Slow Off-Rate Modified Aptamer refers to an aptamer having improved off-rate characteristics. SOMAmers can be generated using the improved SELEX methods described in U.S. Pat. No. 7,947,447, entitled “Method for Generating Aptamers with Improved Off-Rates.” In some embodiments, a slow off-rate aptamer (including an aptamers comprising at least one nucleotide with a hydrophobic modification) has an off-rate (t1/2) of ≥20 minutes ≥30 minutes, ≥60 minutes, ≥90 minutes, ≥120 minutes, ≥150 minutes, ≥ 180 minutes, ≥210 minutes, or ≥240 minutes.

As used herein, an aptamer comprising two different types of 5-position modified pyrimidines or C-5 modified pyrimidines may be referred to as “dual modified aptamers”, aptamers having “two modified bases”, aptamers having “two base modifications” or “two bases modified”, aptamer having “double modified bases”, all of which may be used interchangeably. A library of aptamers or aptamer library may also use the same terminology. Thus, in some embodiments, an aptamer comprises two different 5-position modified pyrimidines wherein the two different 5-position modified pyrimidines are selected from a NapdC and a NapdU, a NapdC and a PPdU, a NapdC and a MOEdU, a NapdC and a TyrdU, a NapdC and a ThrdU, a PPdC and a PPdU, a PPdC and a NapdU, a PPdC and a MOEdU, a PPdC and a TyrdU, a PPdC and a ThrdU, a NapdC and a 2NapdU, a NapdC and a TrpdU, a 2NapdC and a NapdU, and 2NapdC and a 2NapdU, a 2NapdC and a PPdU, a 2NapdC and a TrpdU, a 2NapdC and a TyrdU, a PPdC and a 2NapdU, a PPdC and a TrpdU, a PPdC and a TyrdU, a TyrdC and a TyrdU, a TrydC and a 2NapdU, a TyrdC and a PPdU, a TyrdC and a TrpdU, a TyrdC and a TyrdU, and a TyrdC and a TyrdU. In some embodiments, an aptamer comprises at least one modified uridine and/or thymidine and at least one modified cytidine, wherein the at least one modified uridine and/or thymidine is modified at the 5-position with a moiety selected from a naphthyl moiety, a benzyl moiety, a fluorobenzyl moiety, a tyrosyl moiety, an indole moiety a morpholino moiety, an isobutyl moiety, a 3,4-methylenedioxy benzyl moiety, a benzothiophenyl moiety, and a benzofuranyl moiety, and wherein the at least one modified cytidine is modified at the 5-position with a moiety selected from a naphthyl moiety, a tyrosyl moiety, and a benzyl moiety. In certain embodiments, the moiety is covalently linked to the 5-position of the base via a linker comprising a group selected from an amide linker, a carbonyl linker, a propynyl linker, an alkyne linker, an ester linker, a urea linker, a carbamate linker, a guanidine linker, an amidine linker, a sulfoxide linker, and a sulfone linker. See FIG. 1 for further exemplary linkers that may be used to covalently link a moiety to the 5-position of a pyrimidine.

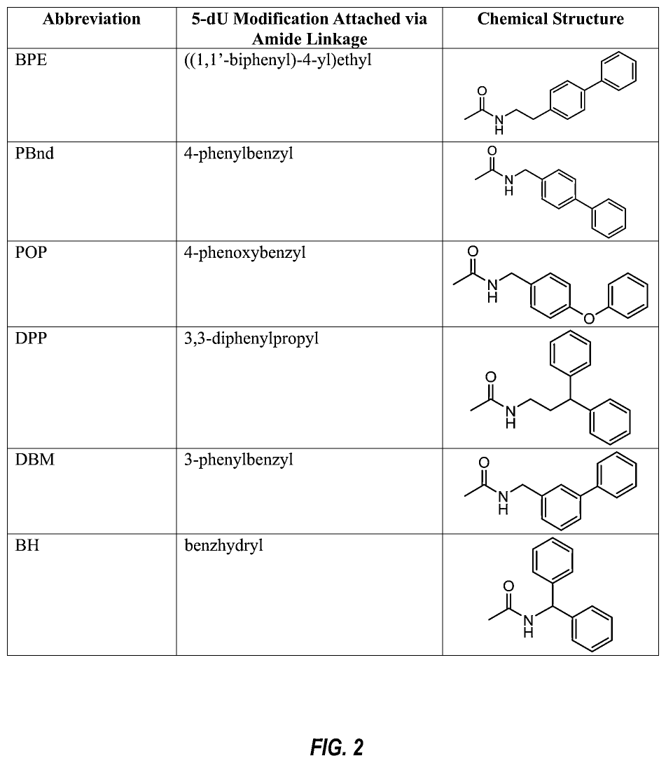

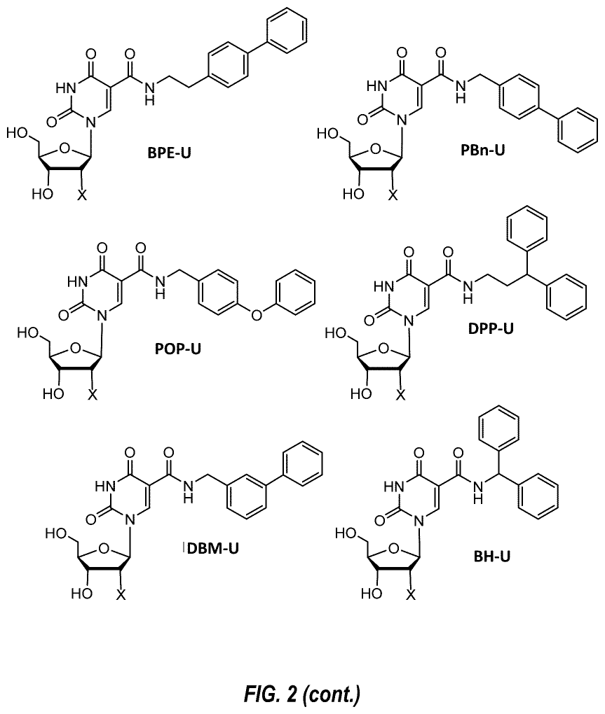

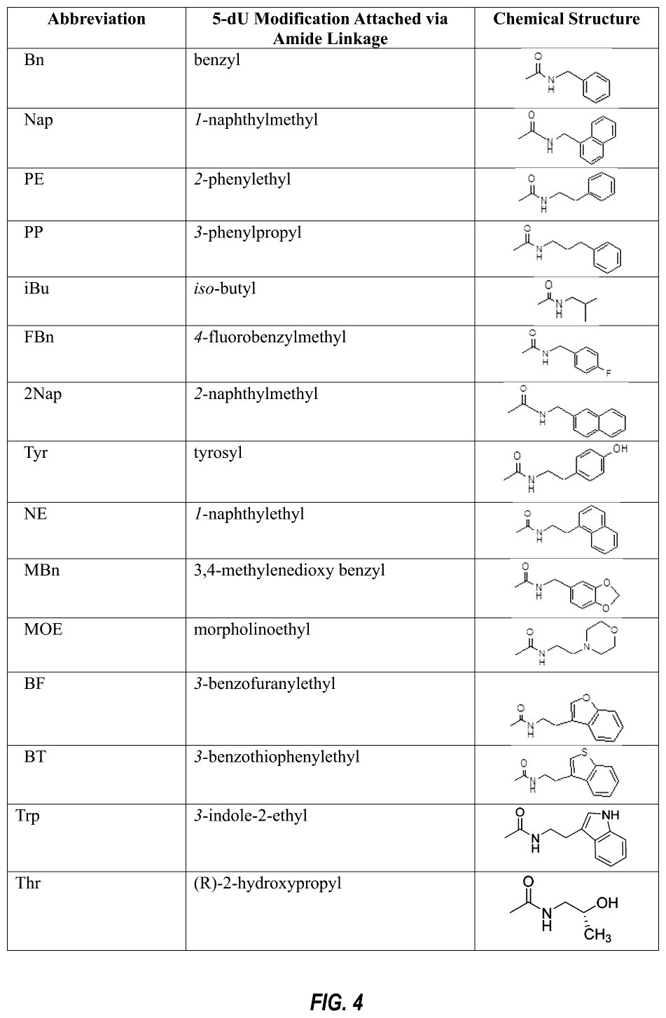

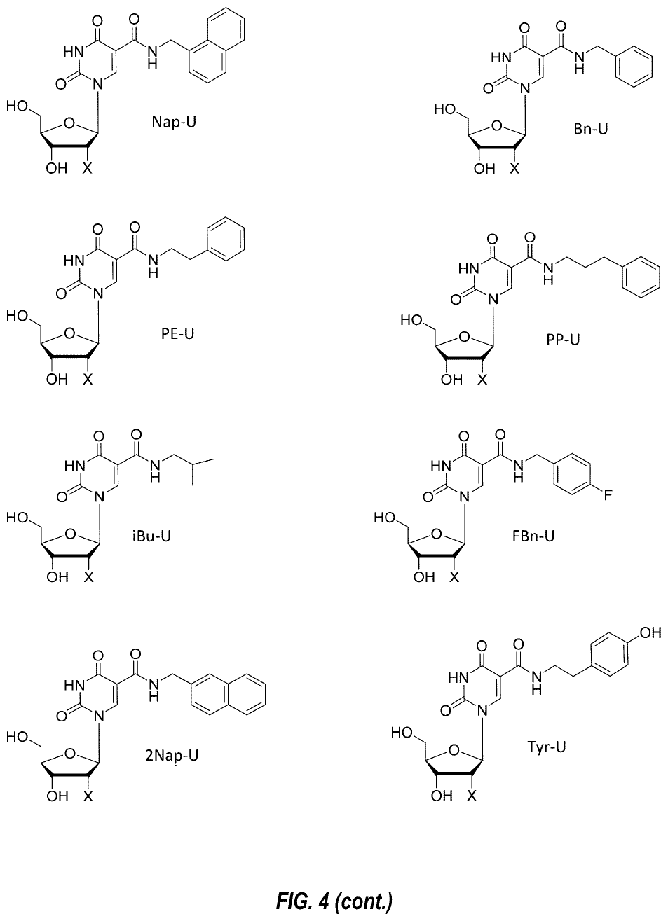

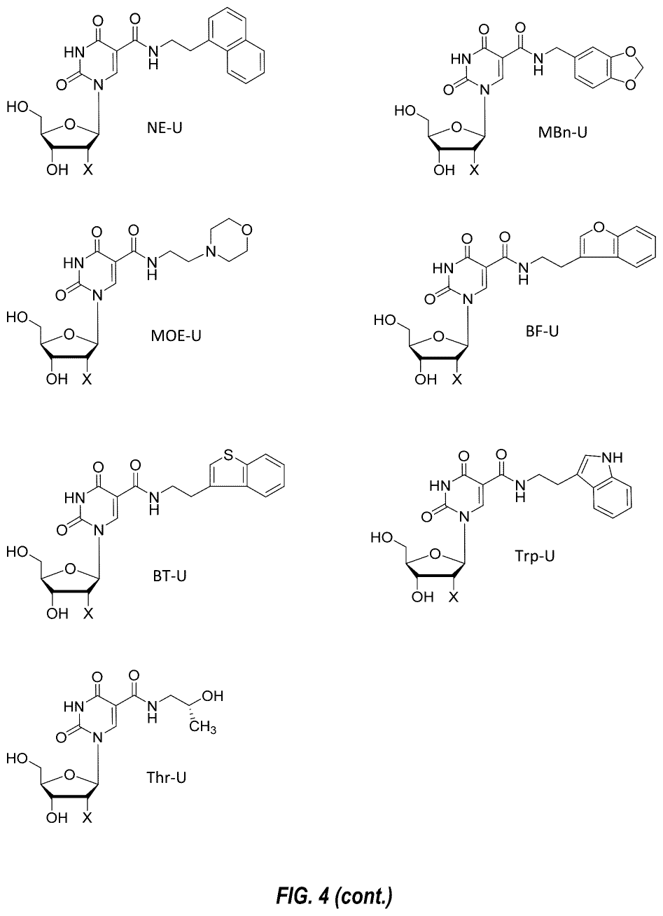

As used herein, a “hydrophobic group” and “hydrophobic moiety” are used interchangeably herein and refer to any group or moiety that is uncharged, a majority of the atoms of the group or moiety are hydrogen and carbon, the group or moiety has a small dipole and/or the group or moiety tends to repel from water. These groups or moieties may comprise an aromatic hydrocarbon or a planar aromatic hydrocarbon. Methods for determining the hydrophobicity or whether molecule (or group or moiety) is hydrophobic are well known in the art and include empirically derived methods, as well as calculation methods. Exemplary methods are described in Zhu Chongqin et al. (2016) Characterizing hydrophobicity of amino acid side chains in a protein environment via measuring contact angle of a water nanodroplet on planar peptide network. Proc. Natl. Acad. Sci., 113 (46) pgs. 12946-12951. As disclosed herein, exemplary hydrophobic moieties included, but are not limited to, Groups I, II, III, IV, V, VII, VIII, IX, XI, XII, XIII, XV and XVI of FIG. 1. Further exemplary hydrophobic moieties include those of FIGS. 2-5 (e.g., BPE. PBnd, POP, DPP, DBM, BH, Bn, Nap, PE, PP, iBu, 2Nap, Try, NE, MBn, BF, BT, Trp).

As used herein, an aptamer comprising a single type of 5-position modified pyrimidine or C-5 modified pyrimidine may be referred to as “single modified aptamers”, aptamers having a “single modified base”, aptamers having a “single base modification” or “single bases modified”, all of which may be used interchangeably. A library of aptamers or aptamer library may also use the same terminology. As used herein, “protein” is used synonymously with “peptide,” “polypeptide,” or “peptide fragment.” A “purified” polypeptide, protein, peptide, or peptide fragment is substantially free of cellular material or other contaminating proteins from the cell, tissue, or cell-free source from which the amino acid sequence is obtained, or substantially free from chemical precursors or other chemicals when chemically synthesized.

In certain embodiments, an aptamer comprises a first 5-position modified pyrimidine and a second 5-position modified pyrimidine, wherein the first 5-position modified pyrimidine comprises a tyrosyl moiety at the 5-position of the first 5-position modified pyrimidine, and the second 5-position modified pyrimidine comprises a naphthyl moiety or benzyl moiety at the 5-position at the second 5-position modified pyrimidine. In a related embodiment the first 5-position modified pyrimidine is a uracil. In a related embodiment, the second 5-position modified pyrimidine is a cytosine. In a related embodiment, at least 10%, 15%, 20%, 25%, 30%, 35%, 40%, 45%, 50%, 55%, 60%, 65%, 70%, 75%, 80%, 85%, 90%, 95%, or 100% of the uracils of the aptamer are modified at the 5-position. In a related embodiment, at least 10%, 15%, 20%, 25%, 30%, 35%, 40%, 45%, 50%, 55%, 60%, 65%, 70%, 75%, 80%, 85%, 90%, 95%, or 100% of the cytosine of the aptamer are modified at the 5-position.

Those of ordinary skill in the art of nucleic acid hybridization will recognize that factors commonly used to impose or control stringency of hybridization include formamide concentration (or other chemical denaturant reagent), salt concentration (i.e., ionic strength), hybridization temperature, detergent concentration, pH and the presence or absence of chaotropes. Optimal stringency for a probe/target sequence combination is often found by the well-known technique of fixing several of the aforementioned stringency factors and then determining the effect of varying a single stringency factor. The same stringency factors can be modulated to thereby control the stringency of hybridization of a PNA to a nucleic acid, except that the hybridization of a PNA is fairly independent of ionic strength. Optimal stringency for an assay may be experimentally determined by examination of each stringency factor until the desired degree of discrimination is achieved.

As used herein, “hybridization,” “hybridizing,” “binding” and like terms, in the context of nucleotide sequences, can be used interchangeably herein. The ability of two nucleotide sequences to hybridize with each other is based on the degree of complementarity of the two sequences, which in turn is based on the fraction of matched complementary nucleotide pairs. The more nucleotides in a given sequence that are complementary to another sequence, the more stringent the conditions can be for hybridization and the more specific will be the binding of the two sequences. Increased stringency is achieved by elevating the temperature, increasing the ratio of co-solvents, lowering the salt concentration, and the like. Hybridization of complementary Watson/Crick base pairs of probes on the microarray and of the target material is generally preferred, but non-Watson/Crick base pairing during hybridization can also occur.

Conventional hybridization solutions and processes for hybridization are described in J. Sambrook, Molecular Cloning: A Laboratory Manual, (supra), incorporated herein by reference. Conditions for hybridization typically include (1) high ionic strength solution, (2) at a controlled temperature, and (3) in the presence of carrier DNA and surfactants and chelators of divalent cations, all of which are known in the art.

As used herein, “array” includes any one, two or three-dimensional arrangement of addressable regions bearing a particular chemical moiety or moieties (for example, biopolymers such peptide nucleic acid molecules, peptides or polynucleotide sequences) associated with that region, where the chemical moiety or moieties are immobilized on the surface in that region. By “immobilized” is meant that the moiety or moieties are stably associated with the substrate surface in the region, such that they do not separate from the region under conditions of using the array, e.g., hybridization and washing and stripping conditions. As is known in the art, the moiety or moieties may be covalently or non-covalently bound to the surface in the region. For example, each region may extend into a third dimension in the case where the substrate is porous while not having any substantial third dimension measurement (thickness) in the case where the substrate is non-porous. An array may contain more than ten, more than one hundred, more than one thousand more than ten thousand features, or even more than one hundred thousand features, in an area of less than 20 cm or even less than 10 cm. For example, features may have widths (that is, diameter, for a round spot) in the range of from about 10 μm to about 1.0 cm. In other embodiments each feature may have a width in the range of about 1.0 μm to about 1.0 mm, such as from about 5.0 μm to about 500 μm, and including from about 10 μm to about 200 μm. Non-round features may have area ranges equivalent to that of circular features with the foregoing width (diameter) ranges. A given feature is made up of chemical moieties, e.g., peptide nucleic acid molecules, peptides, nucleic acids, that bind to (e.g., hybridize to) the target molecule (e.g., target nucleic acid or aptamer), such that a given feature corresponds to a particular target.

The term “biological sample”, “sample”, and “test sample” are used interchangeably herein to refer to any material, biological fluid, tissue, or cell obtained or otherwise derived from an individual, and environmental, animal, or food sample. This includes blood (including whole blood, leukocytes, peripheral blood mononuclear cells, buffy coat, plasma, and serum), sputum, tears, mucus, nasal washes, nasal aspirate, breath, urine, semen, saliva, peritoneal washings, ascites, cystic fluid, meningeal fluid, amniotic fluid, glandular fluid, lymph fluid, nipple aspirate, bronchial aspirate (e.g., bronchoalveolar lavage), bronchial brushing, synovial fluid, joint aspirate, organ secretions, cells, a cellular extract, and cerebrospinal fluid. This also includes experimentally separated fractions of all of the preceding. For example, a blood sample can be fractionated into serum, plasma, or into fractions containing particular types of blood cells, such as red blood cells or white blood cells (leukocytes). In some embodiments, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas, and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual.

“Target” or “target molecule” or “target” refers herein to any compound upon which a nucleic acid can act in a desired or intended manner. In various embodiments, such as a proteomic assay, a target molecule can be a protein, peptide, nucleic acid, carbohydrate, lipid, polysaccharide, glycoprotein, hormone, receptor, antigen, antibody, virus, pathogen, toxic substance, substrate, metabolite, transition state analog, cofactor, inhibitor, drug, dye, nutrient, growth factor, cell, tissue, any portion or fragment of any of the foregoing, etc., without limitation. Virtually any chemical or biological effector may be a suitable target. Molecules of any size can serve as targets. A target can also be modified in certain ways to enhance the likelihood or strength of an interaction between the target and the nucleic acid. A target can also include any minor variation of a particular compound or molecule, such as, in the case of a protein, for example, minor variations in amino acid sequence, disulfide bond formation, glycosylation, lipidation, acetylation, phosphorylation, or any other manipulation or modification, such as conjugation with a labeling component, which does not substantially alter the identity of the molecule. A “target molecule” or “target” is a set of copies of one type or species of molecule or multimolecular structure that is capable of binding to an aptamer. “Target molecules” or “targets” refer to more than one such set of molecules. Embodiments of the SELEX process in which the target is a peptide are described in U.S. Pat. No. 6,376,190, entitled “Modified SELEX Processes Without Purified Protein.” In some embodiments, a target is a protein.

The phrase “oligonucleotide bound to a surface of a solid support” or “probe bound to a solid support” refers to an oligonucleotide that is immobilized on a surface of a solid substrate, where the substrate can have a variety of configurations, e.g., a sheet, bead, particle, slide, wafer, web, fiber, tube, capillary, microfluidic channel or reservoir, or other structure. In certain embodiments, the collections of oligonucleotides employed herein are present on a surface of the same planar support, e.g., in the form of an array. Immobilization of oligonucleotides on a substrate or surface can be accomplished by well-known techniques, commonly available in the literature. See for example A. C. Pease, et al., Proc. Nat. Acad. Sci, USA, 91:5022-5026 (1994); Z. Guo, et al., Nucleic Acids Res, 22, 5456-65 (1994); and M. Schena, et al., Science, 270, 467-70 (1995), each incorporated by reference herein.

Synthesis of polynucleotides is described in detail, for example, in Caruthers, Science 230:281-285, 1985; Itakura et al., Ann. Rev. Biochem. 53:323-356; Hunkapillar et al., Nature 310:105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. Nos. 4,458,066, 4,500,707, 5,153,319, 5,869,643, EP 0294196, and elsewhere. The phosphoramidite and phosphite triester approaches are most broadly used, but other approaches include the phosphodiester approach, the phosphotriester approach and the H-phosphonate approach. The substrates are typically functionalized to bond to the first deposited monomer. Suitable techniques for functionalizing substrates with such linking moieties are described, for example, in Southern, E. M., Maskos, U. and Elder, J. K., Genomics, 13, 1007-1017, 1992. In the case of array fabrication, different monomers and activator may be deposited at different addresses on the substrate during any one cycle so that the different features of the completed array will have different desired biopolymer sequences. One or more intermediate further steps may be required in each cycle, such as the conventional oxidation, capping and washing steps in the case of in situ fabrication of polynucleotide arrays (again, these steps may be performed in flooding procedure).

A computer program may be utilized to carry out one or more steps of any of the methods disclosed herein. Another aspect of the present disclosure is a computer program product comprising a computer readable storage medium having a computer program stored thereon which, when loaded into a computer, performs or assists in the performance of any of the methods disclosed herein.

One aspect of the disclosure is a product of any of the methods disclosed herein, namely, an assay result, which may be evaluated at the site of the testing or it may be shipped to another site for evaluation and communication to an interested party at a remote location, if desired. As used herein, “remote location” refers to a location that is physically different than that at which the results are obtained. Accordingly, the results may be sent to a different room, a different building, a different part of city, a different city, and so forth. The data may be transmitted by any suitable means such as, e.g., facsimile, mail, overnight delivery, e-mail, ftp, voice mail, and the like.

“Communicating” information refers to the transmission of the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data.

### Exemplary Surface Hybridization Catalysts

As used herein, a “surface hybridization catalyst” or “hybridization catalyst” refers to any material that catalyzes the hybridization of a solution-phase oligonucleotide, such as an aptamer (e.g., a SOMAmer), to a solid-phase oligonucleotide, which is an oligonucleotide bound to a solid support, such as an array. In some embodiments, a hybridization catalyst is hydrophilic and cationic. In some embodiments, a hybridization catalyst comprises a main chain (or “backbone”) with side chains bound to the main chain, often forming bottle brush structures. In some embodiments, the hybridization catalyst comprises a polymeric cationic amino acid as the main chain and polyethylene glycol (PEG) as the side chain. Nonlimiting examples of such hybridization catalysts include catalysts comprising polylysine (PLL), polyarginine, or polyornithine as the main chain. In some embodiments, the hybridization catalyst comprises side chains that are neutral and water-soluble. Nonlimiting examples of such side chains include polyethylene glycol, polyacrylamide, polysaccharides, polyvinyl alcohol, dextran, and derivatives thereof. The side chain may be attached to various positions of the main chain. As a nonlimiting example, the hybridization catalyst polylysine-graft-polyethylene glycol (PLL-g-PEG) may comprise PEG side chains attached to the amine moieties of the polylysine main chain. Alternatively, polylysine-graft-polyethylene glycol (PLL-g-PEG) may comprise PEG side chains attached to alkyl moieties of the polylysine main chain.

The molecular weight of the hybridization catalyst can be controlled, in various embodiments, by the length of the main chain, length of the side chain, or grafting density of the side chain. For example, PLL200-g10-PEG5k contains 200 lysines per molecule with 10% PEG modification, or 20 PEG groups per molecule, with each PEG group having a molecular weight of 5 kDa. The following table shows exemplary PLL-g-PEG hybridization catalysts, the number of lysines per molecule, percentage of PEG modification, and number of PEG groups per molecule. The table also shows the number of amines per molecule, which is equal to the number of lysines per molecule minus the number of PEG groups per molecule, and which is used to calculate the amount of hybridization catalyst to be used based on the desired N:P ratio, as discussed herein.

| | Number of | Percentage | Number of | N/molecule |
|---|---|---|---|---|
| | lysines per | PEG | PEG groups/ | for this |
| Reagent | molecule | modification | molecule | reagent |
| Pll200-g10-PEG5k | 200 | 10 | 20 | 180 |
| Pll200-g20-PEG5k | 200 | 20 | 40 | 160 |
| Pll200-g30-PEG5k | 200 | 30 | 60 | 140 |
| Pll20-g10-PEG5k | 20 | 10 | 2 | 18 |
| Pll20-g20-PEG5k | 20 | 20 | 4 | 16 |
| Pll20-g30-PEG5k | 20 | 30 | 6 | 14 |

In some embodiments, the hybridization catalyst comprises 10-500 or 10-300 or 20-200 lysines in the polylysine main chain. In some embodiments, the hybridization catalyst comprises 5-50% or 5-40% or 5-30% or 10-30% PEG modification. In some embodiments, the PEG has an average molecular weight of 1-20 kDa or 2-10 kDa, or 2-8 kDa, or about 5 kDa.

Various hybridization catalysts may be used in the present methods, including various main chain lengths, side chain lengths, and grafting densities, and the amount of the selected hybridization catalyst to use in a hybridization reaction may be calculated for the desired N:P ratio as provided herein. These factors are not particularly limited and may be appropriately selected and prepared by known methods by a person skilled in the art.

### Exemplary Surface Hybridization

The present methods may be applied to any method involving hybridization of solution-phase nucleic acids to solid-phase nucleic acids. As used herein, “solution-phase nucleic acids” refers to any type of nucleic acid, including but not limited to RNA, DNA, modified oligonucleotides, aptamers, genomic DNA, etc., that is present in solution and not bound to a solid support. A solution-phase nucleic acid may be conjugated to a non-nucleic acid moiety, such as, without limitation, a protein, an antibody, a hormone, a small molecule, etc. As used herein, “solid-phase nucleic acids” refers to any type of nucleic acid, as discussed above, bound to one or more solid supports, including but not limited to, 3-dimensional solid supports such as beads or other particles, and 2-dimensional solid supports such as planar arrays.

“Solid support” refers to any substrate having a surface to which molecules may be attached, directly or indirectly, through either covalent or non-covalent bonds. The solid support may include any substrate material that is capable of providing physical support for the capture elements or probes, such as nucleic acids, that are attached to the surface. The material is generally capable of enduring conditions related to the attachment of the capture elements or probes to the surface and any subsequent treatment, handling, or processing encountered during the performance of an assay. The materials may be naturally occurring, synthetic, or a modification of a naturally occurring material. Suitable solid support materials may include silicon, a silicon wafer chip, graphite, mirrored surfaces, laminates, membranes, ceramics, plastics (including polymers such as, e.g., poly(vinyl chloride), cyclo-olefin copolymers, agarose gels or beads, polyacrylamide, polyacrylate, polyethylene, polypropylene, poly(4-methylbutene), polystyrene, polymethacrylate, poly(ethylene terephthalate), polytetrafluoroethylene (PTFE or Teflon®), nylon, poly(vinyl butyrate)), germanium, gallium arsenide, gold, silver, Langmuir Blodgett films, a flow through chip, etc., either used by themselves or in conjunction with other materials. Additional rigid materials may be considered, such as glass, which includes silica and further includes, for example, glass that is available as Bioglass. Other materials that may be employed include porous materials, such as, for example, controlled pore glass beads, crosslinked beaded Sepharose® or agarose resins, or copolymers of crosslinked bis-acrylamide and azalactone. Other beads include nanoparticles, polymer beads, solid core beads, paramagnetic beads, or microbeads. Any other materials known in the art that are capable of having one or more functional groups, such as any of an amino, carboxyl, thiol, or hydroxyl functional group, for example, incorporated on its surface, are also contemplated.

The material used for a solid support may take any of a variety of configurations ranging from simple to complex. The solid support can have any one of a number of shapes, including a strip, plate, disk, rod, particle, bead, tube, well (microtiter), and the like. The solid support may be porous or non-porous, magnetic, paramagnetic, or non-magnetic, polydisperse or monodisperse, hydrophilic or hydrophobic. The solid support may also be in the form of a gel or slurry of closely-packed (as in a column matrix) or loosely-packed particles. In some embodiments, the solid support is a planar array, beads, or a flow cell.

In some embodiments, a DNA hybridization array, or chip, is used to hybridize each solution-phase nucleic acid to a unique or series of unique probes immobilized on a slide or chip such as Agilent arrays, Illumina BeadChip Arrays, NimbleGen arrays or custom printed arrays. Each unique probe is complementary to a sequence on a solution-phase nucleic acid. The complementary sequence may be a unique hybridization tag incorporated in the solution-phase nucleic acid, or a portion of the solution-phase nucleic acid, or the entire solution-phase nucleic acid. The solution-phase nucleic acids are added to a hybridization buffer comprising a hybridization catalyst provided herein and hybridized as described herein. In some embodiments, the hybridization reaction is carried out for up to 20 hours, or up to 15 hours, or up to 10 hours, or up to 8 hours, or up to 6 hours, or for about 1-10 hours, or about 1-8 hours, or about 1-6 hours, or about 2-10 hours, or about 2-8 hours, or about 2-6 hours, or about 4 hours. The arrays are washed and then scanned in a fluorescent slide scanner, producing an image of the aptamer hybridization intensity on each feature of the array. Image segmentation and quantification is accomplished using image processing software, such as Array Vision. In some embodiments, the hybridization reaction comprises at least 100, at least 500, at least 1000, at least 2000, at least 3000, or at least 5000 different solution-phase nucleic acids having different nucleic acid sequences and/or at least 100, at least 500, at least 1000, at least 2000, at least 3000, or at least 5000 different solid-phase nucleic acids having different nucleic acid sequences.

In one embodiment, addressable micro-beads having unique DNA probes complementary to the solution-phase nucleic acids as described above are used for hybridization. The micro-beads may be addressable with unique fluorescent dyes, such as Luminex beads technology, or use bar code labels as in the Illumina VeraCode technology, or laser powered transponders. In one embodiment, the solution-phase nucleic acids are added to a hybridization buffer comprising a hybridization catalyst provided herein and hybridized as described herein. The solutions are then processed on a Luminex instrument which counts the individual bead types and quantifies the fluorescent signal. In another embodiment, the VeraCode beads are contacted with the solution-phase nucleic acids in a hybridization buffer comprising a hybridization catalyst described herein and then deposited on a gridded surface and scanned using a slide scanner for identification and fluorescence quantification. In some embodiments, the hybridization reaction comprises at least 100, at least 500, at least 1000, at least 2000, at least 3000, or at least 5000 different solution-phase nucleic acids having different nucleic acid sequences and/or at least 100, at least 500, at least 1000, at least 2000, at least 3000, or at least 5000 different solid-phase nucleic acids bound to micro-beads and having different nucleic acid sequences.

In some embodiments, the solution-phase nucleic acids can be amplified and optionally tagged before hybridization. Standard PCR amplification can be used, including but not limited to, quantitative PCR (qPCR) or RT-PCR. Such amplification can be used prior to hybridization.

In various embodiment, the methods provided herein comprise hybridizing a plurality of solution-phase nucleic acids to a plurality of solid-phase nucleic acids, wherein the plurality of solution-phase nucleic acids is at least 100, at least 200, at least 1000, at least 2000, at least 3000, or at least 5000 different nucleic acids, i.e., at least 100, at least 200, at least 1000, at least 2000, at least 3000, or at least 5000 different nucleic acids having different nucleic acid sequences. “Different nucleic acid sequences” as used herein means that each nucleic acid of the at least 100, at least 200, at least 1000, at least 2000, at least 3000, or at least 5000 different nucleic acids differs from each other nucleic acid of the at least 100, at least 200, at least 1000, at least 2000, at least 3000, or at least 5000 different nucleic acids by at least one nucleobase in their respective sequences. This includes, for example, nucleic acids of different length and/or different sequence.

In some embodiments, including a hybridization catalyst when hybridizing solution-phase nucleic acids to solid-phase nucleic acids may decrease the time-to-answer by facilitating faster hybridization and/or increasing the amount of hybridization. In some embodiments, including a hybridization catalyst when hybridizing solution-phase nucleic acids to solid-phase nucleic acids may decrease the limit-of-detection. “Faster” hybridization, in some embodiments, means that the same amount or extent of hybridization occurs in a shorter period of time and/or an increased amount or extent of hybridization occurs in the same period of time.

In some embodiments, the amount of hybridization catalyst to include in a surface hybridization reaction is determined by the ratio of the number of cations in the hybridization catalyst (“N”) to the total number of phosphates in the solution-phase nucleic acids and solid-phase nucleic acids (“P”). The inventors surprisingly found that when determining the amount of hybridization catalyst to include in the hybridization reaction, the number of phosphates in not just the solution-phase nucleic acids, but also the solid-phase nucleic acids, should be considered. Accordingly, for hybridization to a planar microarray, for example, the number of phosphates in the nucleic acids bound to the microarray is calculated and added to the number of phosphates in the solution-phase nucleic acids, and the amount of hybridization catalyst determined accordingly. An exemplary calculation for a microarray hybridization reaction, wherein the microarray comprises 65,000 features and a probe density of 1×10¹³, and the hybridization catalyst is PLL200-g20-PEG5k, follows:

| Property | Value | Unit |
|---|---|---|
| Hybridization catalyst | PLL200-g20-PEG5k | 20% grafted |
| Desired N:P ratio | 100 | |
| Diameter of each feature on the microarray | 36 | μm |
| Radius of each feature on the microarray | 18 | μm |
| Area of each feature on the microarray | 1017.36 | μm² |
| Number of features on the microarray | 65000 | |
| Total active area on the microarray surface | 66128400 | μm² |
| | 0.661284 | cm² |
| Density of probes on the microarray surface | 1.00E+13 | probes/cm² |
| Probes per microarray surface | 6.61E+12 | |
| Amount of DNA on the microarray surface | 1.10E−11 | moles of DNA on |
| | | microarray surface |
| | 11 | pmol |
| Approximate concentration of each oligonucleotide | 10 | pM/oligonucleotide |
| in solution | | |
| Number of different oligonucleotides in solution | 7300 | |
| Total concentration of oligonucleotides in solution | 73000 | pM |
| Assay volume | 50 | μl |
| Total amount of oligonucleotides in solution | 3.65 | pmol |
| Total amount of nucleic acids in the experiment | 1.47E+01 | pmol |
| Average length of the nucleic acids | 60 | nucleotides |
| Total number of phosphates in the experiment | 8.79E+02 | pmol |
| Molecular weight of the hybridization catalyst | 226 | kDa |
| Number of lysines per monomer of hybridization | 200 | |
| catalyst | | |
| Percent PEG modifications | 20 | % |
| Number of PEG groups per monomer of hybridization | 40 | |
| catalyst | | |
| Number of unscreened nitrogens (N) per monomer | 160 | |
| of hybridization catalyst | | |
| Amount of N for an N:P ratio of 100 | 8.79E+04 | pmol |
| Amount of hybridization catalyst to achieve | 5.49E+02 | pmol |
| amount N | | |
| Concentration of the hybridization catalyst to | 1.10E+01 | μM |
| achieve N:P ratio of 100 | | |

As shown above, including only the solution-phase oligonucleotides in calculating the desired N:P ratio would result in hybridization catalyst concentration of only 25% of the concentration calculated above (about 2.7 μM versus 11 μM). Stated another way, the present methods take into consideration the number of phosphates in the solid-phase nucleic acids in addition to the phosphates in the solution-phase nucleic acids. The inventors surprisingly found that including only the phosphates in the solution-phase nucleic acids in calculating the amount of hybridization catalyst needed to achieve a desired N:P ratio can result in insufficient concentrations of hybridization catalyst and inadequate improvement in hybridization. Surprisingly, the present inventors found that including the phosphates of the solid-phase nucleic acids in determining the amount of hybridization catalyst to include, results in significantly improved hybridization rate and/or extent.

### Exemplary Assays Involving Solid-Phase Hybridization

In various embodiments, the hybridization catalysts may be used in one or more hybridization steps in any assay method involving hybridization of solution-phase nucleic acids to solid-phase nucleic acids, and for example any assay method comprising multiplex solid-phase hybridization. Nonlimiting exemplary assays that may include multiplex solid-phase hybridization include next-generation sequencing, diagnostic microarrays (such as those for detecting specific genetic mutations, chromosomal aberrations, epigenetic changes, SNPs, etc.), gene expression microarrays, microsatellite analysis, strand-displacement assays, and the like.

In various embodiments, the solution-phase nucleic acids are polynucleotides, including but not limited to deoxyribonucleic acid (DNA), ribonucleic acid (RNA), modified oligonucleotides, which may be based on DNA, RNA, and/or analogs thereof, including double-stranded polynucleotides. It is understood that the term “solution-phase nucleic acid” does not refer to or infer a specific length of the nucleic acid. The term “nucleic acid” encompasses polynucleotides, oligonucleotides of any length and sequence. In various embodiments, solution-phase nucleic acids may be genomic DNA, cDNA, RNA (including mRNA, microRNA, tRNA, etc.), mixed RNA/DNA, modified oligonucleotides, or aptamers. A solution-phase nucleic acid may be conjugated to a non-nucleic acid moiety, such as, without limitation, a protein, an antibody, a lipid, a small molecule, etc. In some such embodiments, the solution-phase nucleic acid is a barcode.

A plurality of solution-phase nucleic acids as described herein refers to at least two solution-phase nucleic acids that differ in sequence. Solution-phase nucleic acid may comprise any type of nucleic acid suitable for use with processes of the disclosure, such as solution-phase nucleic acid that can hybridize to solid phase nucleic acid, for example. A solution-phase nucleic in certain embodiments can comprise DNA (e.g., complementary DNA (cDNA), genomic DNA (gDNA), mitochondrial DNA (mtDNA) and the like), RNA (e.g., messenger RNA (mRNA), short inhibitory RNA (siRNA), ribosomal RNA (rRNA), tRNA and the like), DNA or RNA analogs (e.g., containing base analogs, sugar analogs and/or a non-native backbone and the like), modified oligonucleotides, and/or aptamers. A nucleic acid can be in any form useful in the hybridization assays provided herein (e.g., linear, circular, supercoiled, single-stranded, double-stranded and the like). A nucleic acid may be, or may be from, a plasmid, phage, autonomously replicating sequence (ARS), centromere, artificial chromosome, chromosome, a cell, a cell nucleus, or cytoplasm of a cell in certain embodiments.

In some embodiments, a hybridization catalyst is used in one or more hybridization steps of a solid phase sequencing assay. In some embodiments, the sequencing assay may be a Next Generation Sequencing (NGS) or massively parallel signature sequencing (MPSS) assay. The solution-phase nucleic acids may be DNA or RNA. In some embodiments, a DNA library may be sequenced from DNA or RNA starting samples. In some such embodiments, the DNA or RNA may be fragmented to prepare the library. An adaptor ligation step may be applied to the cDNA before a PCR step with primers that allow for subsequent universal PCR. PCR may be performed with target-specific primers complementary to universal PCR primers to amplify one or more genes of interest. In some embodiments, amplifying a cDNA library with universal primers facilitates certain hybridization-based capture methods. In some embodiments, target primers may be used with tags and/or in combination with universal primers. In some embodiments, multiplex PCR may be used.

Following PCR, the amplified nucleic acids may be sequenced using the surface hybridization catalysts described herein in the hybridization step of the sequencing reaction. The sequencing may include dual index sequencing to increase the number of samples per run. The sequencing may include whole genome sequencing, genotyping, gene expression and/or transcriptome profiling, and/or detection of epigenetic changes. The sequencing may be performed using a sequencing platform, for example, high throughput sequencing such as NGS or MPSS. Commercial high-throughput sequencing platforms include those from Illumina, Qiagen, or ThermoFisher Scientific.

In some embodiments, the hybridization catalysts provided herein may be used in RNA sequencing, such as RNA-Seq. RNA for use in the sequencing assay may be isolated from a sample using standard methods for extraction. Briefly, in some embodiments, RNA-Seq methods use reverse transcriptase to convert RNA to cDNA using either random primers or oligo (dT) primers. The RNA is fragmented or poly(a) selected and then fragmented, followed by random priming, oligo (T) priming, or random hexamer priming. First and second strand cDNAs are synthesized and the cDNA is amplified for sequencing. The sample is then sequenced using the hybridization catalysts described herein in the hybridization step of the sequencing reaction. Additional types of RNA sequencing may also be used with the surface hybridization catalysts described herein.

In some embodiments, the hybridization catalysts provided herein may be used in assays involving strand-displacement where one of the nucleic acids in the strand-displacement assay is bound to a solid surface. In such an assay a first oligonucleotide bound to a solid surface is hybridized to a second oligonucleotide. A third oligonucleotide is introduced in a hybridization buffer comprising a hybridization catalyst, where the third oligonucleotide is capable of hybridizing to the first oligonucleotide and displacing (thus releasing) the second oligonucleotide. An exemplary assay that involves strand displacement on a solid surface is described, for example, in PCT Publication No. WO 2022/060728 A1.

In some embodiments, the hybridization assays provided herein are conducted in a hybridization buffer that comprises a hybridization catalyst. Any suitable hybridization buffer may be used, to which can be added the hybridization catalyst in the desired amount. In some embodiments, the hybridization buffer comprises a monovalent salt. In some embodiments, the hybridization buffer comprises sodium citrate and the monovalent salt is sodium. The sodium citrate concentration can range from 10 mM to 1 M, or 50-500 mM, or 50-400 mM, or 100-300 mM. The hybridization buffer can further comprise a surfactant, such as a nonionic surfactant polysorbate 20 or tween 20. The polysorbate 20 or tween 20 concentration can range from 0.1 wt % to 2 wt % or 0.1 wt % to 1 wt %. In some embodiments, a hybridization buffer comprises a metal chelator, such as EDTA. In some embodiments, the assays herein are performed in an Agilent hybridization buffer (Agilent, part number 5190-0403) to which is added a hybridization catalyst.

### Exemplary Proteomics Assays

Multiplexed aptamer assays in solution-based target interaction and separation steps are described, e.g. in U.S. Pat. Nos. 7,855,054 and 7,964,356 and PCT Application PCT/US2013/044792. In one embodiment, a multiplex assay is described herein at Example 1.

In a multiplex assay format where multiple target proteins are being measured by multiple capture reagents, the natural variation in the abundance of the different target proteins can limit the ability of certain capture reagents to measure certain target proteins (e.g., high abundance target proteins may saturate the assay and prevent or reduce the ability of the assay to measure low abundance target proteins). To address this variation in the biological sample, the aptamer reagents may be separated into at least two different groups (Capture Reagents for DIL1 and Capture Reagents for DIL2), preferably three different groups (A3—Capture Reagents for DIL1; A2—Capture Reagents for DIL2 and A1—Capture Reagents for DIL3), based on the abundance of their respective protein target in the biological sample. Each of the capture reagent groups, A1, A2 and A3 each have a different set of aptamers, with the aptamers having specific affinity for a target protein. The biological sample is diluted into two (Dilution 1 or DIL1 and Dilution 2 or DIL2), preferably three, different dilution groups (Dilution 1 or DIL1; Dilution 2 or DIL2 and Dilution 3 or DIL3) to create separate test samples based on relative concentrations of the protein targets to be detected by their capture reagents. Thus, the biological sample is diluted into high, medium and low abundant target protein dilution groups, where the least abundant protein targets are measured in the least diluted group, and the most abundant protein targets are measured in the greatest diluted group. The capture reagents for their respective dilution groups are incubated together (e.g., the A3 set of aptamers are incubated with the test sample of Dilution 1 or DIL1; the A2 set of aptamers are incubated with the test sample of Dilution 2 or DIL2 and the A1 set of aptamers are incubated with the test sample of Dilution 3 or DIL3). The total number of aptamers for A1, A2 and A3 may be 4,000; 4,500; 5,000 or more aptamers.

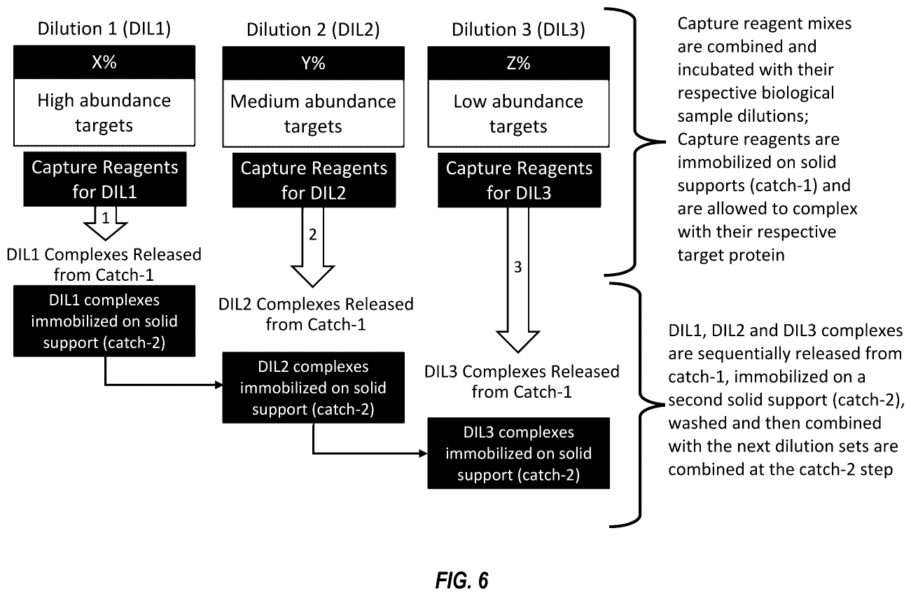

FIG. 6 provides an example overview of the dilution sets for a biological sample, the corresponding capture reagent sets for their respective dilutions, and the general overview of the sequential two-catch system (catch-1 and catch-2). Three different dilution groups may be created from a biological sample that includes a Z % dilution of the biological sample or DIL3, a Y % dilution of the biological sample or DIL2 and a X % dilution of the biological sample or DIL1, where Z is greater than Y, and Y is greater than X (or Z is a greater dilution than the Y dilution, and the Y dilution is a greater dilution than the X dilution). Each dilution has its own set of corresponding capture reagents (A3 for DIL1, A2 for DIL2 and A1 for DIL3) that bind to a specific set of proteins.

In some embodiments, the present disclosure describes methods to perform aptamer- and photoaptamer-based multiplexed assays for the quantification of one or more target molecule(s) that may be present in a test sample wherein the aptamer (or photoaptamer) can be separated from the aptamer-target affinity complex (or photoaptamer-target covalent complex) for final detection using a hybridization-based nucleic acid detection method that comprises a hybridization catalyst provided herein

As used herein “Catch-1” refers to the partitioning of an aptamer-target affinity complex or aptamer-target covalent complex. The purpose of Catch-1 is to remove substantially all of the components in the test sample that are not associated with the aptamer. Removing the majority of such components will generally improve target tagging efficiency by removing non-target molecules from the target tagging step used for Catch-2 capture and may lead to lower assay background. In one embodiment, a tag is attached to the aptamer either before the assay, during preparation of the assay, or during the assay by appending the tag to the aptamer. In one embodiment, the tag is a releasable tag. In one embodiment, the releasable tag comprises a cleavable linker and a tag. As described above, tagged aptamer can be captured on a solid support where the solid support comprises a capture element appropriate for the tag. The solid support can then be washed as described herein prior to equilibration with the test sample to remove any unwanted materials (Catch-0).

As used herein “Catch-2” refers to the partitioning of an aptamer-target affinity complex or aptamer-target covalent complex based on the capture of the target molecule. The purpose of the Catch-2 step is to remove free, or uncomplexed, aptamer from the test sample prior to detection and optional quantification. Removing free aptamer from the sample allows for the detection of the aptamer-target affinity or aptamer-target covalent complexes by any suitable nucleic acid detection technique. When using Q-PCR for detection and optional quantification, the removal of free aptamer is needed for accurate detection and quantification of the target molecule.

In one embodiment, the target molecule is a protein or peptide, and free aptamer is partitioned from the aptamer-target affinity (or covalent) complex (and the rest of the test sample) using reagents that can be incorporated into proteins (and peptides) and complexes that include proteins (or peptides), such as, for example, an aptamer-target affinity (or covalent) complex. The tagged protein (or peptide) and aptamer-target affinity (or covalent) complex can be immobilized on a solid support, enabling partitioning of the protein (or peptide) and the aptamer-target affinity (or covalent) complex from free aptamer. Such tagging can include, for example, a biotin moiety that can be incorporated into the protein or peptide.

In one embodiment, a Catch-2 tag is attached to the protein (or peptide) either before the assay, during preparation of the assay, or during the assay by chemically attaching the tag to the targets. In one embodiment the Catch-2 tag is a releasable tag. In one embodiment, the releasable tag comprises a cleavable linker and a tag. It is generally not necessary, however, to release the protein (or peptide) from the Catch-2 solid support. As described above, tagged targets can be captured on a second solid support where the solid support comprises a capture element appropriate for the target tag. The solid support is then washed with various buffered solutions including buffered solutions comprising organic solvents and buffered solutions comprising salts and/or detergents containing salts and/or detergents.

After washing the second solid support, the aptamer-target affinity complexes are then subject to a dissociation step in which the complexes are disrupted to yield free aptamer while the target molecules generally remain bound to the solid support through the binding interaction of the capture element and target capture tag. The aptamer can be released from the aptamer-target affinity complex by any method that disrupts the structure of either the aptamer or the target. This may be achieved though washing of the support bound aptamer-target affinity complexes in high salt buffer which dissociates the non-covalently bound aptamer-target complexes. Eluted free aptamers are collected and detected. In another embodiment, high or low pH is used to disrupt the aptamer-target affinity complexes. In another embodiment high temperature is used to dissociate aptamer-target affinity complexes. In another embodiment, a combination of any of the above methods may be used. In another embodiment, proteolytic digestion of the protein moiety of the aptamer-target affinity complex is used to release the aptamer component.

Exemplary proteomic affinity assays (multiplex assays) are described, for example, in PCT Publication No. WO 2019/246289.

In the case of aptamer-target covalent complexes, release of the aptamer for subsequent quantification is accomplished using a cleavable linker in the aptamer construct. In another embodiment, a cleavable linker in the target tag will result in the release of the aptamer-target covalent complex.

Released aptamers may be quantified using a hybridization-based method described herein. In some embodiments, released aptamers are quantified on an Agilent 8×15k microarray slide in Agilent HiRPM hybridization buffer containing an amount of hybridization catalyst calculated as described herein.

Following hybridization, the microarray slides may be imaged with a microarray scanner (such as an Agilent G2565CA Microarray Scanner System, Agilent Technologies). The resulting tiff images may be processed using Agilent feature extraction software version 10.7.3.1 with the GEI_107_Sep09 protocol.

As has been described above, one object of a proteomic assay is to convert a protein signal into an aptamer signal. As a result, the quantity of aptamers collected/detected is indicative of, and may be directly proportional to, the quantity of target molecules bound and to the quantity of target molecules in the sample. In addition to the following embodiments of detection methods, other detection methods will be known to one skilled in the art.

Some detection methods require an explicit label to be incorporated into the aptamer prior to detection. In these embodiments, labels, such as, for example, fluorescent or chemiluminescent dyes can be incorporated into aptamers either during or post synthesis using standard techniques for nucleic acid synthesis. Radioactive labels can be incorporated either during synthesis or post synthesis using standard enzyme reactions with the appropriate reagents. Labeling can also occur after the Catch-2 partitioning and elution by using suitable enzymatic techniques. For example, using a primer with the above-mentioned labels, PCR will incorporate labels into the amplification product of the eluted aptamers. When using a gel technique for quantification, different size mass labels can be incorporated using PCR as well. These mass labels can also incorporate different fluorescent or chemiluminescent dyes for additional multiplexing capacity. Labels may be added indirectly to aptamers by using a specific tag incorporated into the aptamer, either during synthesis or post synthetically, and then adding a probe that associates with the tag and carries the label. The labels include those described above as well as enzymes used in standard assays for colorimetric readouts, for example. These enzymes work in combination with enzyme substrates and include enzymes such as, for example, horseradish peroxidase (HRP) and alkaline phosphatase (AP). Labels may also include materials or compounds that are electrochemical functional groups for electrochemical detection.

For example, the aptamer may be labeled, as described above, with a radioactive isotope such as phosphorous 32 (³²P) prior to contacting the test sample. Employing any one of the four basic assays, and variations thereof as discussed above, aptamer detection may be simply accomplished by quantifying the radioactivity on the second solid support at the end of the assay. The counts of radioactivity will be directly proportional to the amount of target in the original test sample. Similarly, labeling an aptamer with a fluorescent dye, as described above, before contacting the test sample allows for a simple fluorescent readout directly on the second solid support. A chemiluminescent label or a quantum dot can be similarly employed for direct readout from the second solid support, requiring no aptamer elution.

For multiplexed detection of aptamers released from the Catch-2 second solid support, a single fluorescent dye, incorporated into each aptamer as described above, can be used with a quantification method that allows for the identification of the aptamer sequence along with quantification of the aptamer level. Methods include but are not limited to DNA chip hybridization, micro-bead hybridization, next generation sequencing and CGE analysis.

### EXAMPLES

The following examples are presented in order to more fully illustrate some embodiments of the invention. They should, in no way be construed, however, as limiting the broad scope of the invention. Those of ordinary skill in the art can readily adopt the underlying principles of this discovery to design various compounds without departing from the spirit of the current invention.

### Example 1. Multiplexed Aptamer Analysis of Samples

This example describes a multiplex aptamer assay that can be used to analyze samples and controls.

### Multiplex Aptamer Assay Method

All steps of the multiplex aptamer assay are performed at room temperature unless otherwise indicated.

### Preparation of Aptamer Master Mix Solutions

5272 aptamers are grouped into three unique mixes, Dil1, Dil2 and Dil3, corresponding to the plasma or serum sample dilutions of 20%, 0.5%, and 0.005%, respectively. The assignment of an aptamer to a mix is empirically determined by assaying a dilution series of matching plasma and serum samples with each aptamer and identifying the sample dilution that gives the largest linear range of signal. The segregation of aptamers and mixing with different dilutions of plasma or serum sample (20%, 0.5% or 0.005%) allow the assay to span a 107-fold range of protein concentrations. The stock solutions for aptamer master mix are prepared in HE-Tween buffer (10 mM Hepes, pH 7.5, 1 mM EDTA, 0.05% Tween 20) at 4 nM aptamer each and are stored frozen at −20° C. 4271 aptamers are mixed in Dil1 mix, 828 aptamers in Dil2, and 173 aptamers in Dil3 mix. Before use, stock solutions are diluted in HE-Tween buffer to a working concentration of 0.55 nM aptamer each and aliquoted into individual use aliquots. Before using aptamer master mixes for Catch-0 plate preparation, working solutions are heat-cooled to refold aptamers by incubating at 95° C. for 10 minutes and then at 25° C. for at least 30 minutes before use.

### Catch-0 Plate Preparation

60 μL of Streptavidin Mag Sepharose 10% slurry (GE Healthcare, 28-9857) are combined with 100 μL of the heat-cooled aptamer master mix. The mixture is washed once with 175 μL of the Assay Buffer (40 mM HEPES, pH 7.5, 100 mM NaCl, 5 mM KCl, 5 mM MgCl₂, 1 mM EDTA, 0.05% Tween-20) and then dispensed to each well of a 96-well plate (Thermo Scientific, AB-0769). Plates are incubated for 30 minutes at 25° C. with shaking at 850 rpm on ThermoMixer C shaker (Eppendorf). After 30 min incubation, 6 μL of the MB Block buffer (50 mM D-Biotin in 50 mM Tris-HCl, pH 8, 0.01% Tween) is added to each well of the plate and the plates are further incubated for 2 min with shaking. Plates are then washed with 175 μL of the Assay Buffer, wash cycle of 1 min shaking on the ThermoMixer C at 850 rpm, followed by separation on the magnet for 30 seconds. After the wash solution is removed, beads are resuspended in 175 μL of Assay buffer and stored at −20° C. until use.

### Catch-2 Bead Preparation

Before the start of the robotic processing of the assay, 10 mg/mL bead slurry of MyOne Streptavidin C1 beads (Dynabeads, part number 35002D, Thermo Scientific) used for Catch-2 step of the multiplex aptamer assay is washed in bulk once with the MB Prep buffer (10 mM Tris-HCl, pH8, 1 mM EDTA, 0.4% SDS) for 5 min followed by two washes with Assay buffer. After the last wash, beads are resuspended at 10 mg/mL concentration, and 75 μL of bead slurry is dispensed into each well of the Catch-2 plate. At the beginning of the assay, Catch-2 plate is placed in the aluminum adapter and placed in the appropriate position on the Fluent deck.

### Sample Thawing and Dilutions

65 μL aliquots of 100% plasma or serum samples, stored in Matrix tubes at −80° C., are thawed by incubating at room temperature for ten minutes. To facilitate thawing, the tubes are placed on top of the fan unit, which circulates the air through the Matrix tube rack. After thawing, the samples are centrifuged at 1000×g for 1 min and placed on the Fluent robot deck for sample dilution. A 20% sample solution is prepared by transferring 35 μL of thawed sample into 96-well plates containing 140 μL of the appropriate sample diluent. Sample diluent for plasma is 50 mM Hepes, pH 7.5, 100 mM NaCl, 8 mM MgCl₂, 5 mM KCl, 1.25 mM EGTA, 1.2 mM Benzamidine, 37.5 μM Z-Block and 1.2% Tween-20. Serum sample diluent contains 75 μM Z-block, and the other components are the same concentration as in the plasma sample diluent. Subsequent dilutions to make 0.5% and 0.005% diluted samples are made into Assay Buffer using serial dilutions on Fluent robot. To make 0.5% sample dilution, intermediate dilution of 20% sample to 4% is made by mixing 45 μL of 20% sample with 180 μL of Assay Buffer, then 0.5% sample is made by mixing 25 μL of 4% diluted sample with 175 μL of Assay Buffer. To make 0.005% sample, 0.05% intermediate dilution is made by mixing 20 μL of 0.5% sample with 180 μL of Assay Buffer, then 0.005% sample is made by mixing 20 μL of 0.05% sample with 180 μL of Assay Buffer.

### Sample Binding Step

Catch-O plates are prepared by immobilizing the aptamer mixes on the Streptavidin Magnetic Sepharose beads as described above. Frozen plates are thawed for 30 min at 25° C. and are washed once with 175 μL of Assay Buffer. 100 μL of each sample dilution (20%, 0.5% and 0.005%) are added to the plates containing beads with three different aptamer master mixes (Dil1, Dil2, and Dil3, respectively). Catch-0 plates are then sealed with aluminum foil seals (Microseal ‘F’ Foil, Bio-Rad) and placed in the 4-plate rotating shakers (PHMP-4, Grant Bio) set at 850 rpm, 28° C. Sample binding step is performed for 3.5 hours.

### Multiplex Aptamer Assay Processing on Fluent Robot

After the sample binding step is complete, Catch-0 plates are placed into aluminum plate adapters and placed on the robot deck. Magnetic bead wash steps are performed using a temperature-controlled plate. For all robotic processing steps, the plates are set at 25° C. temperature except for Catch-2 washes as described below. Plates are washed 4 times with 175 μL of Assay Buffer, each wash cycle is programmed to shake the plates at 1000 rpm for at least 1 min followed by separation of the magnetic beads for at least 30 seconds before buffer aspiration. During the last wash cycle, the Tag reagent is prepared by diluting 100× Tag reagent (EZ-Link NHS-PEG4-Biotin, part number 21363, Thermo, 100 mM solution prepared in anhydrous DMSO) 1:100 in the Assay buffer and poured in the trough on the robot deck. 100 μL of Tag reagent is added to each of the wells in the plates and incubated with shaking at 1200 rpm for 5 min to biotinylate proteins captured on the bead surface. Biotinylation reactions are quenched by addition of 175 μL of Quench buffer (20 mM glycine in Assay buffer) to each well. Plates are incubated static for 3 min, then washed 4 times with 175 μL of Assay buffer, and washes are performed under the same conditions as described above.

### Photo-Cleavage and Kinetic Challenge

After the last wash of the plates, 90 μL of Photocleavage buffer (2 μM of an oligonucleotide competitor in Assay buffer; the competitor has a nucleotide sequence of 5′-(AC-Bn-Bn) 7-AC-3′, where Bn indicates a 5-position benzyl-substituted deoxyuridine residue) is added to each well of the plates. The plates are moved to a photocleavage substation on the Fluent deck. The substation consists of a BlackRay light source (UVP XX-Series Bench Lamps, 365 nm) and three Bioshake 3000-T shakers (Q Instruments). Plates are irradiated for 20 min with shaking at 1000 rpm.

### Catch-2 Bead Capture

At the end of the photocleavage process, the buffer is removed from Catch-2 plate via magnetic separation, and the plate is washed once with 100 μL of Assay buffer. Photo-cleaved eluate containing aptamer-protein complexes is removed from each Catch-0 plate starting with the dilution 3 plate. All 90 μL of the solution is first transferred to the Catch-1 Eluate plate positioned on the shaker with raised magnets to trap any Streptavidin Magnetic Sepharose beads that might have been aspirated. After that, the solution is transferred to the Catch-2 plate, and the plate is incubated for 3 min with shaking at 1400 rpm at 25° C. After the incubation for 3 min, the magnetic beads are separated for 90 seconds, the solution is removed from the plate, and the photocleaved Dil2 plate solution is added to plate. Following the identical process, the solution from Dil1 plate is added and incubated for 3 min. At the end of the 3 min incubation, 6 μL of the MB Block buffer is added to the magnetic bead suspension and beads are incubated for 2 min with shaking at 1200 rpm at 25° C. After this incubation, the plate is transferred to a different shaker, which is preset to 38° C. Magnetic beads are separated for 2 minutes before removing the solution. Then, the Catch-2 plate is washed 4 times with 175 μL of MB Wash buffer (20% glycerol in Assay Buffer), where each wash cycle is programmed to shake the beads at 1200 rpm for 1 min and allow the beads to partition on the magnet for 3.5 minutes. During the last bead separation step, the shaker temperature is set to 25° C. Then the beads are washed once with 175 μL of Assay buffer. For this wash step, beads are shaken at 1200 rpm for 1 min and then allowed to separate on the magnet for 2 minutes. Following the wash step, aptamers are eluted from the purified aptamer-protein complexes using Elution buffer (1.8 M NaCl₀₄, 40 mM PIPES, pH 6.8, 1 mM EDTA, 0.05% Triton X-100). Elution is done using 75 μL of Elution buffer for 10 min at 25° C., shaking beads at 1250 rpm. 70 μL of the eluate is transferred to the Archive plate and separated on the magnet to partition any magnetic beads that might have been aspirated. 10 μL of the eluted material is transferred to the black half-area plate, diluted 1:5 in the Assay buffer, and used to measure the Cyanine 3 fluorescence signals, which are monitored as internal assay QC. 20 μL of the eluted material is transferred to the plate containing 5 μL of the Hybridization Blocking solution (Oligo aCGH/ChIP-on-chip Hybridization Kit, Large Volume, Agilent Technologies 5188-5380, containing a spike of Cyanine 3-labeled DNA sequence complementary to the corner marker probes on Agilent arrays). This plate is removed from the robot deck and further processed for hybridization (see below). Archive plate with the remaining eluted solution is heat-sealed using aluminum foil and stored at −20° C.

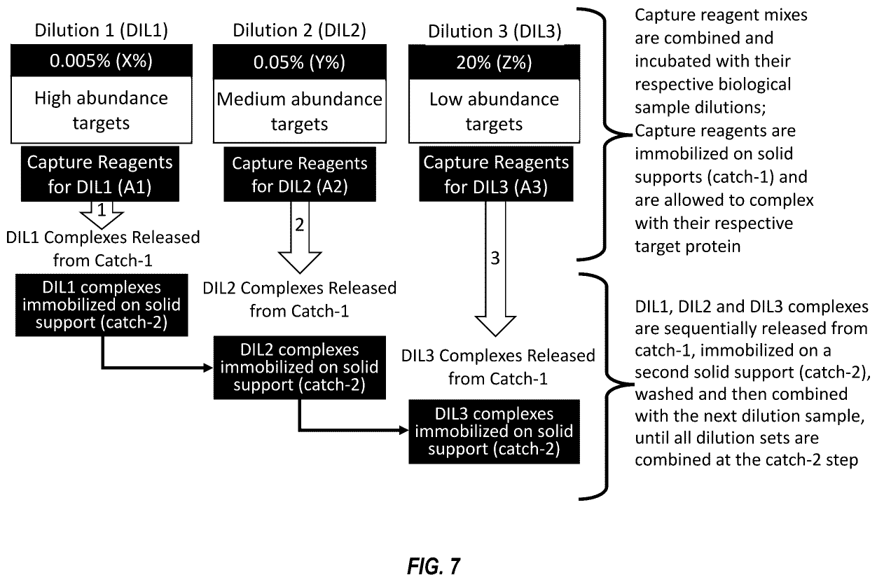

Alternatively, a sequential release and catch of the dilution samples of the biological sample, along with the respective aptamer group, is performed in the course of transfer from the catch-1 phase of the assay to the catch-2 phase of the assay. A general overview of a two dilution and three dilution sequential catch format is shown in FIGS. 8 and 6, respectively. For this example, the same three different dilution groups of plasma are made (DIL3, DIL2, and DIL1), along with the same aptamer groups (A1, A2 and A3), as described above (see FIG. 7). The same approach as described above for the catch-1 phase of the assay is followed; however, for this example, the different dilution groups are released individually and transferred to the catch-2 phase of the assay sequentially instead of at the same time (see FIG. 7). More specifically, for Condition 1, the DIL1 group that is incubated with aptamer group A1 (DIL1-A1 group) is released from catch-1, immobilized onto a second solid support (catch-2), and washed. Next, the DIL2 group that is incubated with aptamer group A2 is released from catch-1, combined with the DIL1-A1 group that is already immobilized on catch-2, and then immobilized onto a second solid support (catch-2). The DIL3 group that is incubated with aptamer group A3 is released from catch-1, immobilized into a second solid support (catch-2), and washed.

### Standard Operating Procedure (SOP) Hybridization

25 μL of 2× Agilent Hybridization buffer (Oligo aCGH/ChIP-on-chip Hybridization Kit, Agilent Technologies, part number 5188-5380) is manually pipetted to each well of the plate containing the eluted samples and blocking buffer. 40 μL of this solution is manually pipetted into each “well” of the hybridization gasket slide (Hybridization Gasket Slide-8 microarrays per slide format, Agilent Technologies). Custom SurePrint G3 8×60k Agilent microarray slides containing 10 probes per array complementary to each aptamer are placed onto the gasket slides according to the manufacturer's protocol. Each assembly (Hybridization Chamber Kit-SureHyb enabled, Agilent Technologies) is tightly clamped and loaded into a hybridization oven for 19 hours at 55° C. rotating at 20 rpm.

### Hybridization in the Presence of Hybridization Catalyst

25 μL of 2× Agilent Hybridization buffer (Oligo aCGH/ChIP-on-chip Hybridization Kit, Agilent Technologies, part number 5188-5380) containing the indicated amount of hybridization catalyst is manually pipetted to each well of the plate containing the eluted samples and blocking buffer. 40 μL of this solution is manually pipetted into each “well” of the hybridization gasket slide (Hybridization Gasket Slide-8 microarrays per slide format, Agilent Technologies). Custom SurePrint G3 8×60k Agilent microarray slides containing 10 probes per array complementary to each aptamer are placed onto the gasket slides according to the manufacturer's protocol. Each assembly (Hybridization Chamber Kit-SureHyb enabled, Agilent Technologies) is tightly clamped and loaded into a hybridization oven for 19 hours at 55° C. rotating at 20 rpm.

### Post-Hybridization Washing

Slide washing is performed using Little Dipper Processor (model 650C, Scigene). Approximately 700 mL of Wash Buffer 1 (Oligo aCGH/ChIP-on-chip Wash Buffer 1, Agilent Technologies) is poured into a large glass staining dish and used to separate microarray slides from the gasket slides. Once disassembled, the slides are quickly transferred into a slide rack in a bath containing Wash Buffer 1 on the Little Dipper. The slides are washed for five minutes in Wash Buffer 1 with mixing via magnetic stir bar. The slide rack is then transferred to the bath with 37° C. Wash Buffer 2 (Oligo aCGH/ChIP-onchip Wash Buffer 2, Agilent Technologies) and allowed to incubate for five minutes with stirring. The slide rack is slowly removed from the second bath and then transferred to a bath containing acetonitrile and incubated for five minutes with stirring.

### Microarray Imaging

The microarray slides are imaged with a microarray scanner (Agilent G4900DA Microarray Scanner System, Agilent Technologies) in the Cyanine 3-channel at 3 μm resolution at 100% PMT setting and the 20-bit option enabled. The resulting tiff images are processed using Agilent Feature Extraction software (version 10.7.3.1 or higher) with the GE1_1200_Jun14 protocol.

### Example 2. Surface Hybridization

Solutions comprising a total of about 7,000 aptamers (SOMAmers), each at a concentration of 10 pM were prepared. These solutions were incubated on the surface of SomaArray slides (Agilent slides comprising 65,000 features and including probes complementary to each of the SOMAmers in the assay) in Agilent hybridization buffer spiked with varying amounts of a hybridization catalyst (PLL200-g20-PEG5k) for 4 hours at room temperature. The negative control comprised the same assay conditions but without the hybridization catalyst. SOMAmers with an N:P ratio (ratio of the number of amines on the catalyst to the number of phosphates on the nucleic acid backbone) of 1, 10, 50, and 100 were examined. The performance of the hybridization catalyst was determined by measuring the fluorescence (relative fluorescence units (RFU)) on the cognate binding site on the microarray surface.

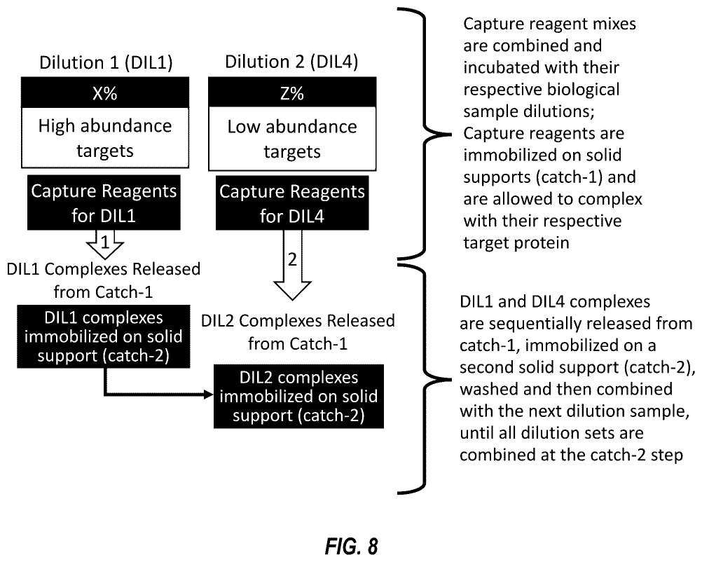

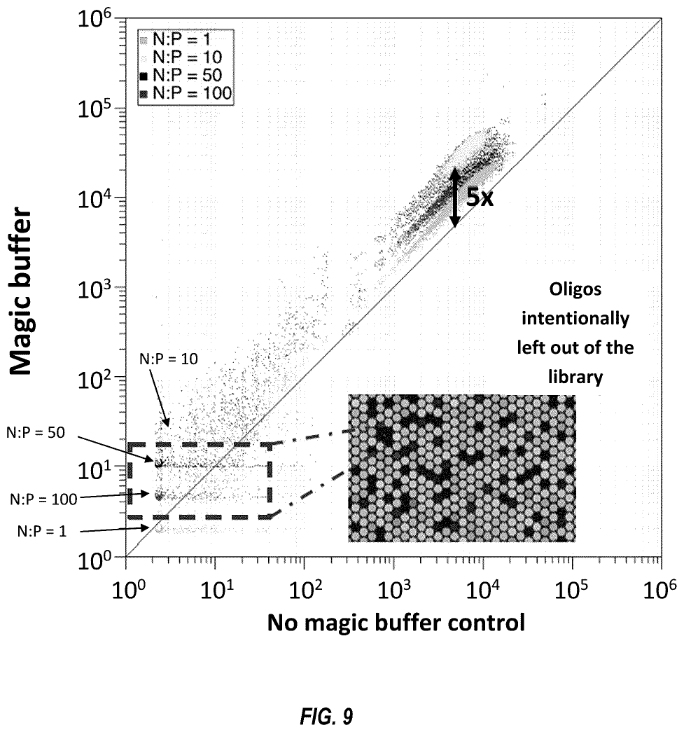

The combined result of the surface hybridization assay of the hybridization catalyst is shown in FIG. 9. FIG. 9 shows increased signals in the presence of the hybridization catalyst (“magic buffer”) compared to the “no hybridization catalyst” condition (“no magic buffer control”), suggesting increased hybridization rate in the presence of hybridization catalyst. Various ratios of the number of amines on the catalyst to the number of phosphates on the nucleic acid backbone (N (amine):P (phosphate) ratio) of the SOMAmer were assessed (ratios 1, 10, 50, and 100). In FIG. 9, the X-axis corresponds to fluorescence (RFU) of the no-catalyst condition and the Y-axis corresponds to fluorescence (RFU) of the four N:P experimental conditions with the catalyst. Each dot represented a single SOMAmer. A line along X=Y indicates identical performance with or without the catalyst with zero acceleration. The Y-offset of the line indicates an A-fold acceleration.

At an N:P ratio of 1:1, A was roughly 2. With an increasing amount of hybridization catalyst, a plateau in performance was reached: A reached a peak of roughly 5 at N:P ratios of 10:1 and 50:1. At a higher N:P ratio, N:P=100, performance was reduced, and A was below 3 in this experiment. The linear nature of the data indicated good concordance with the no-hybridization catalyst condition. The spread of the points around the hypothetical line along each N:P data set was a little larger than that for a self-concordance, indicating some sequence dependence may be present. However, this effect was not significant as, for instance, RFUs were spread out over less than a factor of 2 for N:P of 10. The absolute background rose somewhat for N:P of 10 and 50 and fell for N:P of 100. The coefficient of variation (CV) for slide positions for which no SOMAmers were added, which are expected to measure zero, were significantly decreased.

### Example 3. Measurement of Coefficient of Variation

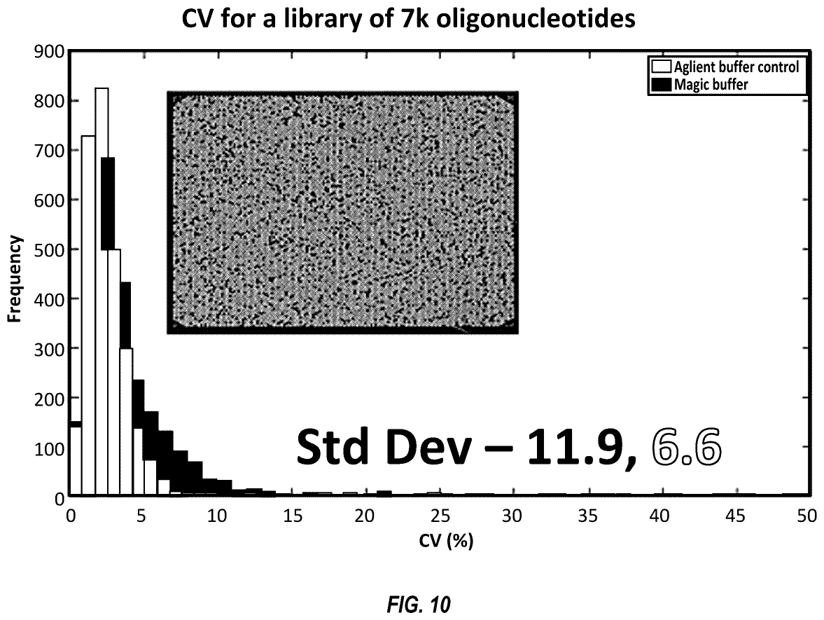

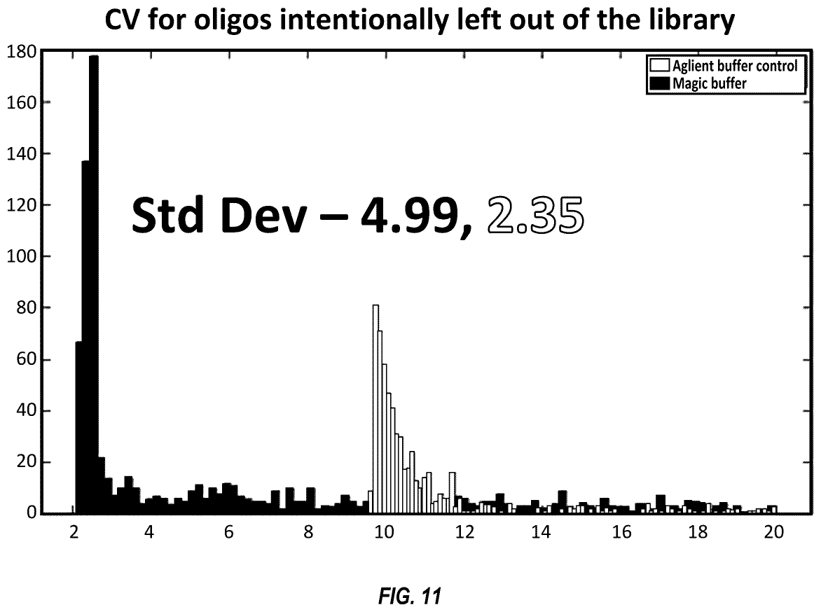

Using the data from the experiment described in Example 2, performance of the hybridization catalyst was determined by measuring the fluorescence (RFU) on the cognate binding site on the microarray surface. The assay results are shown in FIG. 10 and FIG. 11.

Accelerating the hybridization of SOMAmers to the microarray surface with the hybridization catalyst resulted in increased fluorescence (RFU) per binding spot and reduced coefficient of variation (CV). Assays conducted in the presence of the hybridization catalyst showed a lower CV than assays without the catalyst, along with a lower standard deviation (FIG. 10). The CV was higher for assays conducted with the hybridization catalyst in the negative control experiments (FIG. 11; Y-axis=frequency and X-axis=intensity (RFU)).

### Example 4. Measurement of the Repeatability

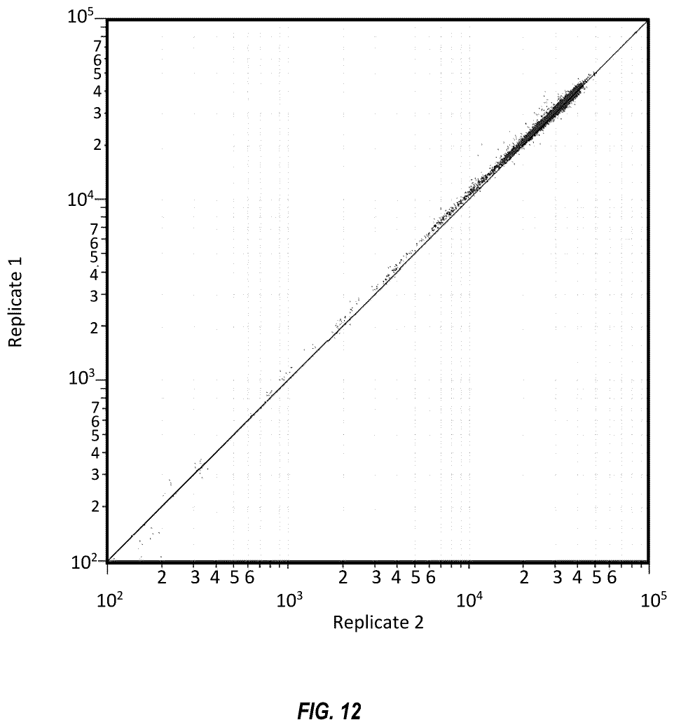

Hybridization of SOMAmers on SomaArray slides with the hybridization catalyst (PLL200-g20-PEG5k) were conducted in replicates, which demonstrated excellent repeatability of the data. See FIG. 12.

### Example 5. Shelf-Life of the Hybridization Catalyst Buffer Solution

A buffer solution comprising PLL200-g20-PEG5k hybridization catalyst was prepared and stored at 4° C. At given experimental time points, a SOMAmer mix was spiked into the aged hybridization catalyst buffer solution, or into freshly prepared hybridization catalyst buffer solution, at an N:P ratio of 10. Relative hybridization performance of the two solutions on Agilent microarray surfaces was measured. The fluorescence (RFU) values recorded on the Agilent scanner was used as a proxy to quantify the differential performance, if any, of the aged versus fresh hybridization catalyst buffer solutions. Experiments were performed at two different SOMAmer library concentrations, ˜10 pM and ˜100 fM, respectively.

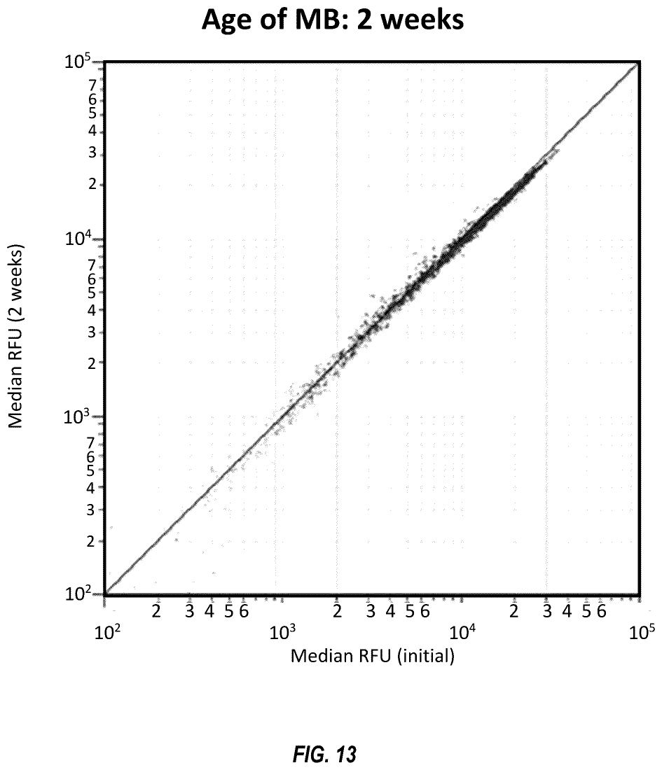

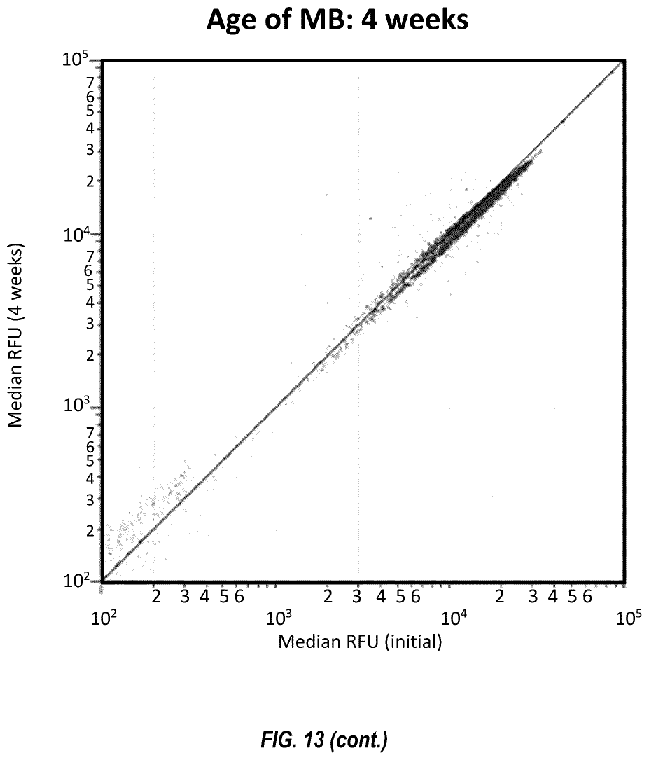

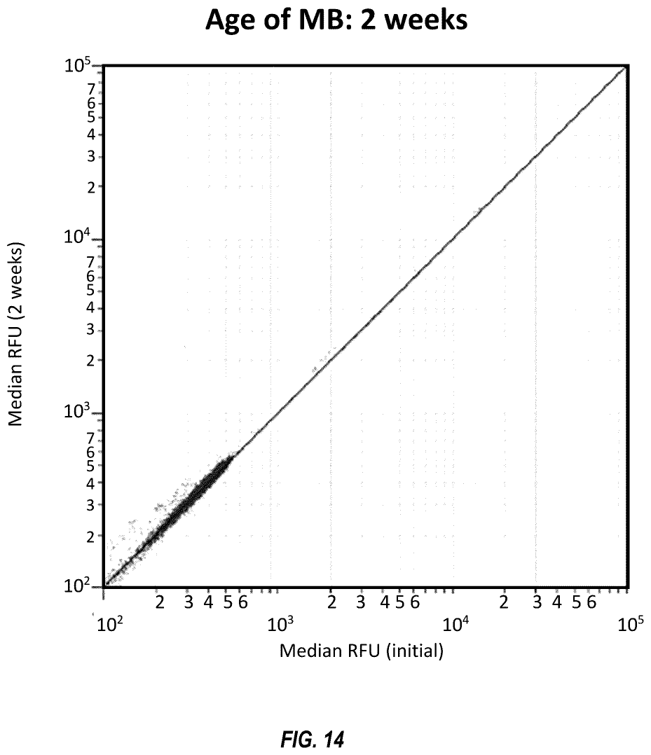

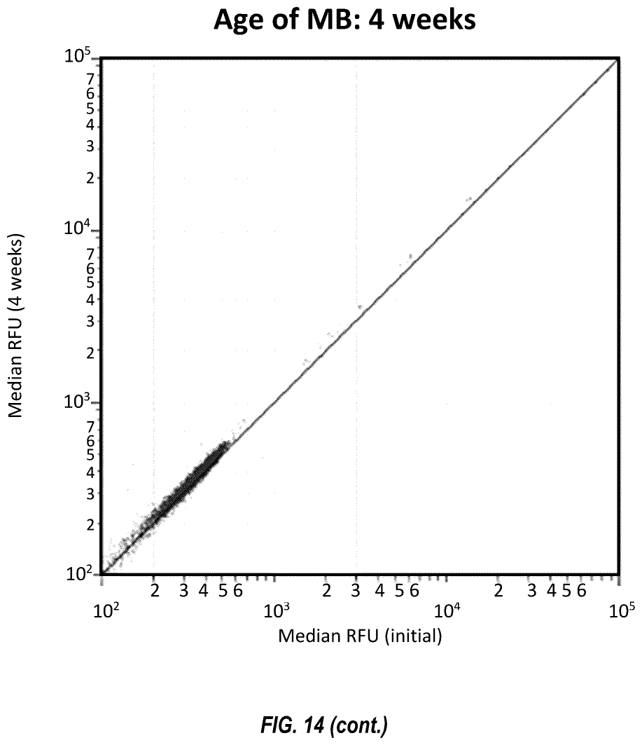

As shown in FIG. 13 and FIG. 14, hybridization of SOMAmers onto Agilent microarray surfaces was not influenced by the age of the hybridization catalyst buffer solution in both high- and low-concentration SOMAmer libraries. The hybridization catalyst buffer solution aged for 2 weeks and 4 weeks showed the same performance. Based on this experiment, the hybridization catalyst buffer solution may be stored at 4° C. for at least 4 weeks.

### Example 6. Measurement of Incubation Times on the Performance of the Hybridization Catalyst Buffer Solution

The microarray hybridization time for SomaScan® assays is typically about 19 hours. As discussed above, accelerated hybridization is observed on microarray surfaces in the presence of hybridization catalyst buffer, allowing the reduction of required incubation time from 19 hours to about 4 hours.

The present experiment was designed to observe the effect of hybridization catalyst buffer solution when the SOMAmer library was incubated for the standard operating procedure (SOP) incubation time of 19 hours. The outcome of this experiment was expected to be influenced by material exhaustion (not enough SOMAmers available in solution once most of them have found their hybridization partner on the surface during the early part of the experiment) as opposed to kinetics as in the 4-hour challenge.

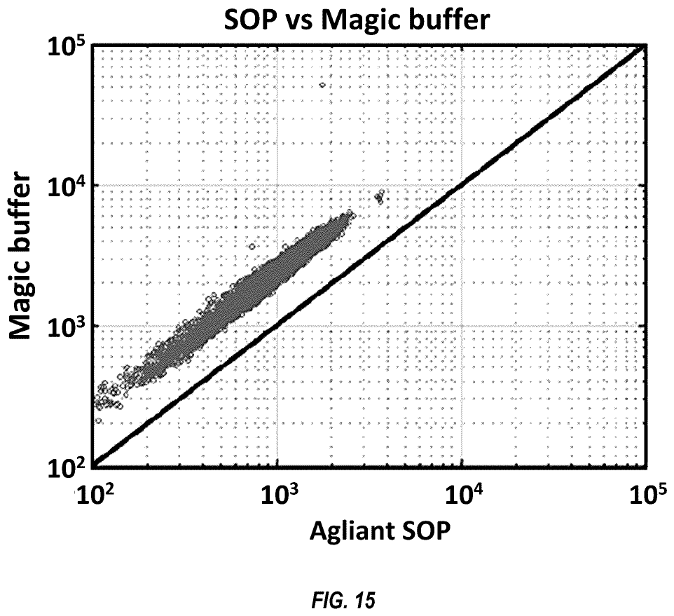

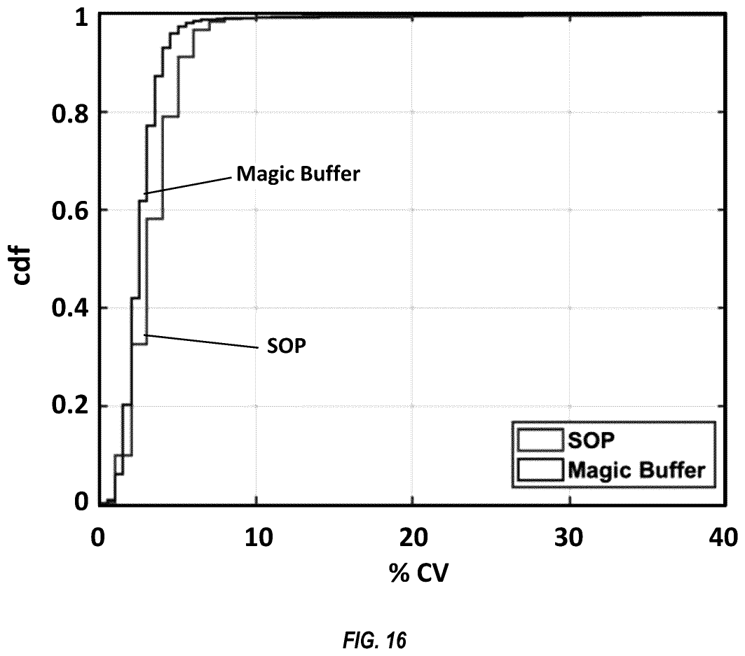

FIG. 15 shows the hybridization performance of the SOMAmer library in the presence of hybridization catalyst buffer solution compared with a control condition (Agilent SOP). At 19-hour incubation, hybridization catalyst buffer solution was shown to result in higher fluorescence signals, in other words, improved hybridization to the arrays, for substantially all SOMAmers in the library. As shown in FIG. 16, analogous to the improvement in CV performance observed for 4-hour incubation assays, an improvement was also observed in 19-hour incubation experiments.

1. A method of hybridizing a plurality of solution-phase nucleic acids to a plurality of solid-phase nucleic acids, comprising contacting the solid-phase nucleic acids with a hybridization solution comprising the solution-phase nucleic acids and a hybridization catalyst, wherein the hybridization catalyst comprises a polycationic main chain with hydrophilic side chains.

2. The method of claim 1, wherein the plurality of solution-phase nucleic acids comprises at least 100, at least 500, at least 1000, at least 2000, at least 3000, or at least 5000 different nucleic acids having different nucleic acid sequences and wherein the plurality of solid-phase nucleic acids comprises at least 100, at least 500, at least 1000, at least 2000, at least 3000, or at least 5000 different nucleic acids having different nucleic acid sequences.

3. (canceled)

4. The method of claim 1, wherein the solid-phase nucleic acids are bound to an array, flow cell, or beads.

5. (canceled)

6. The method of claim 1, wherein the hybridization solution comprises 100-300 mM monovalent cationic salt.

7. (canceled)

8. The method of claim 1, wherein the hybridization catalyst comprises a polylysine, polyarginine, or polyornithine main chain.

9. The method of claim 1, wherein the hybridization catalyst comprises polyethylene glycol, polyacrylamide, polyvinyl alcohol, or dextran side chains.

10. The method of claim 1, wherein the hybridization catalyst comprises a polylysine main chain and polyethylene glycol (PEG) side chains.

11. The method of claim 10, wherein the hybridization catalyst comprises 10-500 or 10-300 or 20-200 lysines in the polylysine main chain; 5-50% or 5-40% or 5-30% or 10-30% PEG modification, and/or wherein the PEG has an average molecular weight of 1-20 kDa or 2-10 kDa, or 2-8 kDa, or about 5 kDa.

12. The method of claim 1, wherein the hybridization catalyst is present in an amount such that the ratio of number of cations in the polycationic backbone of the hybridization catalyst to the number of phosphates in the plurality of solution-phase nucleic acids and plurality of solid-phase nucleic acids (N:P ratio) is between 1:1 and 200:1, or between 1:1 and 100:1, or between 1:1 and 50:1, or between 1:1 and 25:1.

13. The method of claim 1, wherein hybridization of the solution-phase nucleic acids to the solid-phase nucleic acids is at least 2-fold, at least 3-fold, at least 4-fold, or at least 5-fold faster in the presence of the hybridization catalyst than in the absence of the hybridization catalyst.

14. The method of claim 1, wherein the solution-phase nucleic acids comprise RNA, DNA, genomic DNA, mitochondrial DNA, mRNA, microRNA, aptamers, and/or modified oligonucleotides.

15. The method of claim 1, wherein the method of hybridization is a step in an assay selected from next-generation sequencing, diagnostic microarrays, gene expression microarrays, microsatellite analysis, proteomic assays, and strand-displacement assays.

16. The method of claim 1, wherein the solution-phase nucleic acids are conjugated to non-nucleic acid moiety selected from a protein, an antibody, a lipid, and a small molecule.

17. (canceled)

18. The method of claim 1, wherein the solution-phase nucleic acids each comprise a barcode sequence.

19. The method of claim 1, wherein the solution-phase nucleic acids are not amplified prior to hybridization to the solid-phase nucleic acids.

20. The method of claim 1, wherein the solution-phase nucleic acids are amplified prior to hybridization to the solid-phase nucleic acids.

21. (canceled)

22. The method of claim 1, wherein the solution-phase nucleic acids are aptamers.

23. (canceled)

24. The method of claim 22, wherein each aptamer comprises at least one, at least two, at least three, or at least five C-5 modified pyrimidines.

25. (canceled)

26. The method of claim 22, wherein each aptamer is capable of binding to a protein target.

27. (canceled)

28. (canceled)

29. A method of detecting a plurality of protein targets in a biological sample, comprising contacting the biological sample with a plurality of aptamers, wherein each aptamer is a nucleic acid, and wherein each aptamer specifically binds a protein target, to form a plurality of aptamer/protein complexes, releasing the bound aptamers from the aptamer/protein complexes into solution to form a plurality of solution-phase nucleic acids, and hybridizing the plurality of solution-phase nucleic acids to a plurality of solid-phase nucleic acids according to the method of claim 1, and detecting the hybridized solution-phase nucleic acids.

30. (canceled)
